(19) United States
(12) Patent Application Publication
(10) Pub. No.: US 2004/0157744 A1
(43) Pub. Date: Aug. 12, 2004

(54) Substituted fluoroalkoxyphenylsulfonyl-amino(thio)carbonyltriazolino(thi)ones

(76) Inventors: Klaus-Helmut Muller, Dusseldorf (DE); Ernst Rudolf F. Gesing, Erkrath (DE); Joachim Kluth, Langenfield (DE); Mark Wilhelm Drewes, Langenfeld (DE); Peter Dahmen, Neuss (DE); Dieter Feucht, Monheim (DE); Rolf Pontzen, Leichlingen (DE)

Correspondence Address: BAYER CROPSCIENCE LP, Patent Department, 100 BAYER ROAD, PITTSBURGH, PA, 15205-9741, US

(21) Appl. No.: 10/474,184

(22) PCT Filed: Mar. 27, 2002
(86) PCT No.: PCT/EP02/03404

(30) Foreign Application Priority Data
Apr. 9, 2001 (DE) 101 17 673.2

(51) Int. Cl. A01N 43/00; C07D 249/12

(52) U.S. Cl. 504/273; 548/263.2

(57) ABSTRACT

The invention relates to substituted fluoroalkoxyphenylsulphonylamino(thio)-carbonyl-triazolin(ethi)ones of the general formula (I)

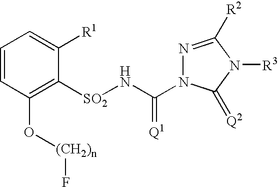

in which the radicals n, Q¹, Q², R¹, R² and R³ are as defined in the description (except for the prior-art compounds 2-(2-fluoro-ethoxy)-6-methyl-N-[(4-methyl-5-oxo-3-propoxy-4,5-dihydro-1H-1,2,4-triazol-1-yl)-carbonyl]-benzenesulphonamide, 2-(2-fluoro-ethoxy)-6-methyl-N-[(4-methyl-5-oxo-3-i-propoxy-4,5-dihydro-1H-1,2,4-triazol-1-yl)-carbonyl]-benzenesulphonamide, 2-(2-fluoro-ethoxy)-6-methyl-N-[4-cyclopropyl-3-methoxy-5-oxo-4,5-dihydro-1H-1,2,4-triazol-1-yl)-carbonyl]-benzenesulphonamide, 2-(2-fluoro-ethoxy)-6-methyl-N-[4-cyclopropyl-5-oxo-3-i-propoxy-4,5-dihydro-1H-1,2,4-triazol-1-yl)-carbonyl]-benzenesulphonamide, 2-(2-fluoro-ethoxy)-6-methyl-N-[3-methoxy-4-methyl-5-oxo-4,5-dihydro-1H-1,2,4-triazol-1-yl)-carbonyl]-benzenesulphonamide, 2-(2-fluoro-ethoxy)-6-methyl-N-[3-ethoxy-4-methyl-5-oxo-4,5-dihydro-1H-1,2,4-triazol-1-yl)-carbonyl]-benzenesulphonamide and 2-(2-fluoro-ethoxy)-6-methyl-N-[3,4-dimethyl-5-oxo-4,5-dihydro-1H-1,2,4-triazol-1-yl)-carbonyl]-benzenesulphonamide (cf. WO-A-97/03056), which are excluded by disclaimer), and to a plurality of processes for their preparation and to their use as herbicides.

The invention relates to novel substituted fluoroalkoxyphenylsulphonylamino(thio)-carbonyl-triazolin(ethi)ones, to processes for their preparation and to their use as plant treatment agents, in particular as herbicides.

It is already known that certain substituted phenylsulphonylaminocarbonyl-triazolinones, such as, for example, the compounds 2-(2-fluoro-ethoxy)-6-methyl-N-[(4-methyl-5-oxo-3-propoxy-4,5-dihydro-1H-1,2,4-triazol-1-yl)-carbonyl]-benzenesulphonamide, 2-(2-fluoro-ethoxy)-6-methyl-N-[(4-methyl-5-oxo-3-1-propoxy-4,5-dihydro-1H-1,2,4-triazol-1-yl)-carbonyl]-benzenesulphonamide, 2-(2-fluoro-ethoxy)-6-methyl-N-[4-cyclopropyl-3-methoxy-5-oxo-4,5-dihydro-1H-1,2,4-triazol-1-yl)-carbonyl]-benzenesulphonamide, 2-(2-fluoro-ethoxy)-6-methyl-N-[4-cyclopropyl-5-oxo-3-i-propoxy-4,5-dihydro-1H-1,2,4-triazol-1-yl)-carbonyl]-benzenesulphonamide, 2-(2-fluoro-ethoxy)-6-methyl-N-[3-methoxy-4-methyl-5-oxo-4,5-dihydro-1H-1,2,4-triazol-1-yl)-carbonyl]-benzenesulphonamide, 2-(2-fluoro-ethoxy)-6-methyl-N-[3-ethoxy-4-methyl-5-oxo-4,5-dihydro-1H-1,2,4-triazol-1-yl)-carbonyl]-benzenesulphonamide and 2-(2-fluoro-ethoxy)-6-methyl-N-[3,4-dimethyl-5-oxo-4,5-dihydro-1H-1,2,4-triazol-1-yl)-carbonyl]-benzenesulphonamide (cf. WO-A-97/03056) have herbicidal properties (cf. also EP-A-341 489, EP-A-422 469, EP-A-425 948, EP-A-431 291, EP-A-507 171, EP-A-534 266). However, the herbicidal activity and the compatibility of these compounds with crop plants are not entirely satisfactory.

This invention, accordingly, provides the novel substituted fluoroalkoxyphenylsulphonylamino(thio)carbonyl-triazolin(ethi)ones of the general formula (I)

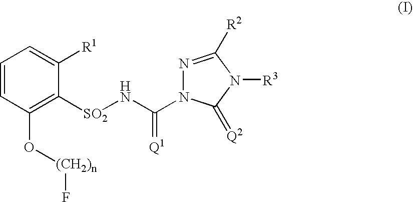

in which

n represents the numbers 2, 3 or 4,

Q¹ represents O (oxygen) or S (sulphur),

Q² represents O (oxygen) or S (sulphur),

R¹ represents in each case optionally cyano-, fluorine-, chlorine-, bromine-, methoxy-, ethoxy-, n- or i-propoxy-substituted methyl, ethyl, n- or i-propyl, n-, s- or t-butyl, methoxy, ethoxy, n- or i-propoxy, n-, i-, s- or t-butoxy, methoxycarbonyl, ethoxycarbonyl, n- or i-propoxycarbonyl,

R² represents hydrogen, cyano, fluorine, chlorine, bromine, represents in each case optionally cyano-, fluorine-, chlorine-, methoxy-, ethoxy-, n- or i-propoxy-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethyl amino, n- or i-propylamino, n-, i-, s- or t-butylamino, methoxyamino, ethoxyamino, n- or i-propoxyamino, n-, i-, s- or t-butoxyamino, represents dimethylamino, diethyl-amino, N-methyl-methoxyamino or N-methyl-ethoxyamino, represents in each case optionally cyano-, fluorine-, chlorine- or bromine-substituted ethenyl, propenyl, butenyl, ethinyl, propinyl, butinyl, propenyloxy, butenyloxy, propinyloxy, butinyloxy, propenylthio, butenylthio, propinylthio, butinylthio, propenylamino, butenylamino, propinylamino or butinylamino, or represents in each case optionally cyano-, fluorine-, chlorine-, bromine-, methyl- or ethyl-substituted cyclopropyl, cyclobutyl, cyclopentyl, cyclohexyl, cyclopropyloxy, cyclobutyloxy, cyclopentyloxy, cyclohexyloxy, cyclopropylamino, cyclobutylamino, cyclopentylamino, cyclohexylamino, cyclopropylmethyl, cyclobutylmethyl, cyclopentylmethyl, cyclohexylmethyl, cyclopropylmethoxy, cyclobutylmethoxy, cyclopentylmethoxy, cyclohexylmethoxy, cyclopropylmethylthio, cyclobutylmethylthio, cyclopentylmethylthio, cyclohexylmethylthio, cyclopropylmethylamino, cyclobutylmethylamino, cyclopentylmethylamino or cyclohexylmethylamino, and

R³ represents hydrogen, represents in each case optionally cyano-, fluorine-, chlorine-, methoxy-, ethoxy-, n- or i-propoxy-substituted methyl, ethyl, n- or i-propyl, methoxy, ethoxy, n- or i-propoxy, methylamino, ethylamino, n- or i-propylamino, represents dimethylamino or diethylamino, or represents in each case optionally cyano-, fluorine-, chlorine-, bromine-, methyl- or ethyl-substituted cyclopropyl, cyclobutyl, cyclopentyl, cyclohexyl, cyclopropylmethyl, cyclobutylmethyl, cyclopentylmethyl or cyclohexylmethyl,

and also salts of compounds of the formula (I),

except for the prior-art compounds 2-(2-fluoro-ethoxy)-6-methyl-N-[(4-methyl-5-oxo-3-propoxy-4,5-dihydro-1H-1,2,4-triazol-1-yl)-carbonyl]-benzenesulphonamide, 2-(2-fluoro-ethoxy)-6-methyl-N-[(4-methyl-5-oxo-3-1-propoxy-4,5-dihydro-1H-1,2,4-triazol-1-yl)-carbonyl]-benzenesulphonamide, 2-(2-fluoro-ethoxy)-6-methyl-N-[4-cyclopropyl-3-methoxy-5-oxo-4,5-dihydro-1H-1,2,4-triazol-1-yl)-carbonyl]-benzenesulphonamide, 2-(2-fluoro-ethoxy)-6-methyl-N-[4-cyclopropyl-5-oxo-3-i-propoxy-4,5-dihydro-1H-1,2,4-triazol-1-yl)-carbonyl]-benzenesulphonamide, 2-(2-fluoro-ethoxy)-6-methyl-N-[3-methoxy-4-methyl-5-oxo-4,5-dihydro-1H-1,2,4-triazol-1-yl)-carbonyl]-benzenesulphonamide, 2-(2-fluoro-ethoxy)-6-methyl-N-[3-ethoxy-4-methyl-5-oxo-4,5-dihydro-1H-1,2,4-triazol-1-yl)-carbonyl]-benzenesulphonamide and 2-(2-fluoro-ethoxy)-6-methyl-N-[3,4-dimethyl-5-oxo-4,5-dihydro-1H-1,2,4-triazol-1-yl)-carbonyl]-benzenesulphonamide (cf. WO-A-97/03056) which are excluded by disclaimer.

Preferred substituents or ranges of radicals present in the formulae given above and below are described below:

n preferably represents the numbers 2, 3 or 4.

Q¹ preferably represents O (oxygen) or S (sulphur).

Q² preferably represents O (oxygen) or S (sulphur).

R¹ preferably represents in each case optionally cyano-, fluorine-, chlorine-, methoxy- or ethoxy-substituted methyl, ethyl, n- or i-propyl, n- or i-butyl, methoxy, ethoxy, n- or i-propoxy, n- or i-butoxy, methoxycarbonyl, ethoxycarbonyl, n- or i-propoxycarbonyl.

R² preferably represents hydrogen, cyano, fluorine, chlorine, bromine, represents in each case optionally cyano-, fluorine-, chlorine-, methoxy-, ethoxy-, n- or i-propoxy-substituted methyl, ethyl, n- or i-propyl, n-, i- or s-butyl, methoxy, ethoxy, n- or i-propoxy, n-, i- or s-butoxy, methylthio, ethylthio, n- or i-propylthio, n-, i- or s-butylthio, methylamino, ethylamino, n- or i-propylamino, n-, i- or s-butylamino, methoxyamino, ethoxyamino, n- or i-propoxyamino, n-, i- or s-butoxyamino, represents dimethylamino, diethylamino, N-methyl-methoxyamino or N-methyl-ethoxyamino, represents in each case optionally cyano-, fluorine-, chlorine- or bromine-substituted ethenyl, propenyl, butinyl, ethinyl, propinyl, butinyl, propenyloxy, butenyloxy, propinyloxy, butinyloxy, propenylthio, butenylthio, propinylthio, butinylthio, propenylamino, butenylamino, propinylamino or butinylamino, or represents in each case optionally cyano-, fluorine-, chlorine-, bromine-, methyl- or ethyl-substituted cyclopropyl, cyclobutyl, cyclopentyl, cyclopropyloxy, cyclobutyloxy, cyclopentyloxy, cyclopropylamino, cyclobutylamino, cyclopentylamino, cyclopropylmethyl, cyclobutylmethyl, cyclopentylmethyl, cyclopropylmethoxy, cyclobutylmethoxy, cyclopentylmethoxy, cyclopropylmethylthio, cyclobutylmethylthio, cyclopentylmethylthio, cyclopropylmethylamino, cyclobutylmethylamino or cyclopentylmethylamino.

R³ preferably represents hydrogen, represents in each case optionally cyano-, fluorine-, chlorine-, methoxy- or ethoxy-substituted methyl, ethyl, methoxy, ethoxy, methylamino, ethylamino, represents dimethylamino, or represent in each case optionally fluorine-, chlorine- or methyl-substituted cyclopropyl, cyclobutyl, cyclopentyl, cyclopropylmethyl, cyclobutylmethyl or cyclopentylmethyl.

n particularly preferably represents the numbers 2, 3 or 4.

Q¹ particularly preferably represents O (oxygen) or S (sulphur).

Q² particularly preferably represents O (oxygen) or S (sulphur).

R¹ particularly preferably represents in each case optionally fluorine-, chlorine-, methoxy- or ethoxy-substituted methyl, ethyl, n- or i-propyl, methoxy, ethoxy, n- or i-propoxy, methoxycarbonyl or ethoxycarbonyl.

R² particularly preferably represents chlorine, bromine, represents in each case optionally cyano-, fluorine-, chlorine-, methoxy-, ethoxy-, n- or i-propoxy-substituted methyl, ethyl, n- or i-propyl, methoxy, ethoxy, n- or i-propoxy, methylthio, ethylthio, n- or i-propylthio, methylamino, ethylamino, n- or i-propylamino, methoxyamino or ethoxyamino, represents dimethylamino or N-methyl-methoxyamino, represents in each case optionally fluorine- or chlorine-substituted ethenyl, propenyl, ethinyl, propinyl, propenyloxy, propinyloxy, propenylthio, propinylthio, propenylamino or propinylamino, or represents in each case optionally fluorine-, chlorine- or methyl-substituted cyclopropyl, cyclopropyloxy, cyclopropylamino, cyclopropylmethyl, cyclopropylmethoxy, cyclopropylmethylthio or cyclopropylmethylamino.

R³ particularly preferably represents methyl, ethyl, methoxy, ethoxy, dimethylamino, cyclopropyl or cyclopropylmethyl.

A further very particularly preferred group are those compounds of the general formula (I) in which

n represents the number 3 or 4.

The invention furthermore preferably provides the sodium, potassium, magnesium, calcium, ammonium, C₁-C₄-alkyl-ammonium, di-(C₁-C₄-alkyl)-ammonium, tri-(C₁-C₄-alkyl)-ammonium, tetra-(C₁-C₄-alkyl)-ammonium, tri-(C₁-C₄-alkyl)-sulphonium, C₅- or C₆-cycloalkyl-ammonium and di-(C₁-C₂-alkyl)-benzyl-ammonium salts of compounds of the formula (I) in which Q¹, Q², R¹, R² and R³ have the meanings given above as being preferred, in particular the sodium salts.

Examples of the compounds of the general formula (I) according to the invention are listed in the groups below.

Group 1

n=2, Q¹=O (oxygen), Q²=O (oxygen), R¹=ethyl, R³=methyl.

In this case, R² represents, for example, H, cyano, chlorine, bromine, methyl, ethyl, n- or i-propyl, n-, i-, s- or t-butyl, methoxy, ethoxy, n- or i-propoxy, n-, i-, s- or t-butoxy, difluoromethoxy, 2,2,2-trifluoro-ethoxy, methylthio, ethylthio, n- or i-propylthio, n-, i-, s- or t-butylthio, methylamino, ethylamino, n- or i-propylamino, n-, i-, s- or t-butylamino, methoxyamino, ethoxyamino, n- or i-propoxyamino, n-, i- and s-butoxyamino, dimethylamino, diethylamino, N-methyl-methoxyamino, ethenyl, 1-propenyl, 2-propenyl (allyl), 2-propenyloxy (allyloxy), 2-propenylthio (allylthio), 2-propinyloxy (propargyloxy), 2-propinylthio (propargylthio), cyclopropyl, cyclopropylmethyl, cyclopropylmethoxy and cyclopropylmethylthio.

Group 2

n=2, Q¹=O (oxygen), Q²=O (oxygen), R¹=n-propyl, R³=methyl.

In this case, R² has, for example, the meanings given above in group 1.

Group 3

n=2, Q¹=O (oxygen), Q²=O (oxygen), R¹=i-propyl, R³=methyl.

In this case, R² has, for example, the meanings given above in group 1.

Group 4

n=2, Q¹=O (oxygen), Q²=O (oxygen), R¹=methoxy, R³=methyl.

In this case, R² has, for example, the meanings given above in group 1.

Group 5

n=2, Q¹=O (oxygen), Q²=O (oxygen), R¹=ethoxy, R³=methyl.

In this case, R² has, for example, the meanings given above in group 1.

Group 6

n=2, Q¹=O (oxygen), Q²=O (oxygen), R¹=n-propoxy, R³=methyl.

In this case, R² has, for example, the meanings given above in group 1.

Group 7

n=2, Q¹=O (oxygen), Q²=O (oxygen), R¹=i-propoxy, R³=methyl.

In this case, R² has, for example, the meanings given above in group 1.

Group 8

n=2, Q¹=O (oxygen), Q²=O (oxygen), R¹=2-fluoroethoxy, R³=methyl.

In this case, R² has, for example, the meanings given above in group 1.

Group 9

n=2, Q¹=O (oxygen), Q²=O (oxygen), R¹=trifluoromethoxy, R³=methyl.

In this case, R² has, for example, the meanings given above in group 1.

Group 10

n=2, Q¹=O (oxygen), Q²=O (oxygen), R¹=methoxycarbonyl, R³=methyl.

In this case, R² has, for example, the meanings given above in group 1.

Group 11

n=2, Q¹=O (oxygen), Q²=O (oxygen), R¹=ethyl, R³=ethyl.

In this case, R² has, for example, the meanings given above in group 1.

Group 12

n=2, Q¹=O (oxygen), Q²=O (oxygen), R¹=n-propyl, R³=ethyl.

In this case, R² has, for example, the meanings given above in group 1.

Group 13

n=2, Q¹=O (oxygen), Q²=O (oxygen), R¹=i-propyl, R³=ethyl.

In this case, R² has, for example, the meanings given above in group 1.

Group 14

n=2, Q¹=O (oxygen), Q²=O (oxygen), R¹=methoxy, R³=ethyl.

In this case, R² has, for example, the meanings given above in group 1.

Group 15

n=2, Q¹=O (oxygen), Q²=O (oxygen), R¹=ethoxy, R³=ethyl.

In this case, R² has, for example, the meanings given above in group 1.

Group 16

n=2, Q¹=O (oxygen), Q²=O (oxygen), R¹=n-propoxy, R³=ethyl.

In this case, R² has, for example, the meanings given above in group 1.

Group 17

n=2, Q¹=O (oxygen), Q²=O (oxygen), R¹=i-propoxy, R³=ethyl.

In this case, R² has, for example, the meanings given above in group 1.

Group 18

n=2, Q¹=O (oxygen), Q²=O (oxygen), R¹=2-fluoro-ethoxy, R³=ethyl.

In this case, R² has, for example, the meanings given above in group 1.

Group 19

n=2, Q¹=O (oxygen), Q²=O (oxygen), R¹=trifluoromethoxy, R³=ethyl.

In this case, R² has, for example, the meanings given above in group 1.

Group 20

n=2, Q¹=O (oxygen), Q²=O (oxygen), R¹=methoxycarbonyl, R³=ethyl.

In this case, R² has, for example, the meanings given above in group 1.

Group 21

n=2, Q¹=O (oxygen), Q²=O (oxygen), R¹=ethyl, R³=methoxy.

In this case, R² has, for example, the meanings given above in group 1.

Group 22

n=2, Q¹=O (oxygen), Q²=O (oxygen), R¹=n-propyl, R³=methoxy.

In this case, R² has, for example, the meanings given above in group 1.

Group 23

n=2, Q¹=O (oxygen), Q²=O (oxygen), R¹=i-propyl, R³=methoxy.

In this case, R² has, for example, the meanings given above in group 1.

Group 24

n=2, Q¹=O (oxygen), Q²=O (oxygen), R¹=methoxy, R³=methoxy.

In this case, R² has, for example, the meanings given above in group 1.

Group 25

n=2, Q¹=O (oxygen), Q²=O (oxygen), R¹=ethoxy, R³=methoxy.

In this case, R² has, for example, the meanings given above in group 1.

Group 26

n=2, Q¹=O (oxygen), Q²=O (oxygen), R¹=n-propoxy, R³=methoxy.

In this case, R² has, for example, the meanings given above in group 1.

Group 27

n=2, Q¹=O (oxygen), Q²=O (oxygen), R¹=i-propoxy, R³=methoxy.

In this case, R² has, for example, the meanings given above in group 1.

Group 28

n=2, Q¹=O (oxygen), Q²=O (oxygen), R¹=2-fluoro-ethoxy, R³=methoxy.

In this case, R² has, for example, the meanings given above in group 1.

Group 29

n=2, Q¹=O (oxygen), Q²=O (oxygen), R¹=trifluoromethoxy, R³=methoxy.

In this case, R² has, for example, the meanings given above in group 1.

Group 30

n=2, Q¹=O (oxygen), Q²=O (oxygen), R¹=methoxycarbonyl, R³=methoxy.

In this case, R² has, for example, the meanings given above in group 1.

Group 31

n=2, Q¹=O (oxygen), Q²=O (oxygen), R¹=ethyl, R³=ethoxy.

In this case, R² has, for example, the meanings given above in group 1.

Group 32

n=2, Q¹=O (oxygen), Q²=O (oxygen), R¹=n-propyl, R³=ethoxy.

In this case, R² has, for example, the meanings given above in group 1.

Group 33

n=2, Q¹=O (oxygen), Q²=O (oxygen), R¹=i-propyl, R³=ethoxy.

In this case, R² has, for example, the meanings given above in group 1.

Group 34

n=2, Q¹=O (oxygen), Q²=O (oxygen), R¹=methoxy, R³=ethoxy.

In this case, R² has, for example, the meanings given above in group 1.

Group 35

n=2, Q¹=O (oxygen), Q²=O (oxygen), R¹=ethoxy, R³=ethoxy.

In this case, R² has, for example, the meanings given above in group 1.

Group 36

n=2, Q¹=O (oxygen), Q²=O (oxygen), R¹=n-propoxy, R³=ethoxy.

In this case, R² has, for example, the meanings given above in group 1.

Group 37

n=2, Q¹=O (oxygen), Q²=O (oxygen), R¹=i-propoxy, R³=ethoxy.

In this case, R² has, for example, the meanings given above in group 1.

Group 38

n=2, Q¹=O (oxygen), Q²=O (oxygen), R¹=2-fluoro-ethoxy, R³=ethoxy.

In this case, R² has, for example, the meanings given above in group 1.

Group 39

n=2, Q¹=O (oxygen), Q²=O (oxygen), R¹=trifluoromethoxy, R³=ethoxy.

In this case, R² has, for example, the meanings given above in group 1.

Group 40

n=2, Q¹=O (oxygen), Q²=O (oxygen), R¹=methoxycarbonyl, R³=ethoxy.

In this case, R² has, for example, the meanings given above in group 1.

Group 41

n=2, Q¹=O (oxygen), Q²=O (oxygen), R¹=ethyl, R³=propargyloxy.

In this case, R² has, for example, the meanings given above in group 1.

Group 42

n=2, Q¹=O (oxygen), Q²=O (oxygen), R¹=n-propyl, R³=propargyloxy.

In this case, R² has, for example, the meanings given above in group 1.

Group 43

n=2, Q¹=O (oxygen), Q²=O (oxygen), R¹=i-propyl, R³=propargyloxy.

In this case, R² has, for example, the meanings given above in group 1.

Group 44

n=2, Q¹=O (oxygen), Q²=O (oxygen), R¹=methoxy, R³=propargyloxy.

In this case, R² has, for example, the meanings given above in group 1.

Group 45

n=2, Q¹=O (oxygen), Q²=(oxygen), R¹=ethoxy, R³=propargyloxy.

In this case, R² has, for example, the meanings given above in group 1.

Group 46

n=2, Q¹=O (oxygen), Q²=O (oxygen), R¹=n-propoxy, R³=propargyloxy.

In this case, R² has, for example, the meanings given above in group 1.

Group 47

n=2, Q¹=O (oxygen), Q²=O (oxygen), R¹=i-propoxy, R³=propargyloxy.

In this case, R² has, for example, the meanings given above in group 1.

Group 48

n=2, Q¹=O (oxygen), Q²=O (oxygen), R¹=2-fluoro-ethoxy, R³=propargyloxy.

In this case, R² has, for example, the meanings given above in group 1.

Group 49

n=2, Q¹=O (oxygen), Q²=O (oxygen), R¹=trifluoromethoxy, R³=propargyloxy.

In this case, R² has, for example, the meanings given above in group 1.

Group 50

n=2, Q¹=O (oxygen), Q²=O (oxygen), R¹=methoxycarbonyl, R³=propargyloxy.

In this case, R² has, for example, the meanings given above in group 1.

Group 51

n=2, Q¹=O (oxygen), Q²=O (oxygen), R¹=ethyl, R³=dimethylamino.

In this case, R² has, for example, the meanings given above in group 1.

Group 52

n=2, Q¹=O (oxygen), Q²=O (oxygen), R¹=n-propyl, R=dimethylamino.

In this case, R² has, for example, the meanings given above in group 1.

Group 53

n=2, Q¹=O (oxygen), Q²=O (oxygen), R¹=i-propyl, R³=dimethylamino.

In this case, R² has, for example, the meanings given above in group 1.

Group 54

n=2, Q¹=O (oxygen), Q²=O (oxygen), R¹=methoxy, R³=dimethylamino.

In this case, R² has, for example, the meanings given above in group 1.

Group 55

n=2, Q¹=O (oxygen), Q²=O (oxygen), R¹=ethoxy, R³=dimethylamino.

In this case, R² has, for example, the meanings given above in group 1.

Group 56

n=2, Q¹=O (oxygen), Q²=O (oxygen), R¹=n-propoxy, R³=dimethylamino.

In this case, R² has, for example, the meanings given above in group 1.

Group 57

n=2, Q¹=O (oxygen), Q²=O (oxygen), R¹=i-propoxy, R³=dimethylamino.

In this case, R² has, for example, the meanings given above in group 1.

Group 58

n=2, Q¹=O (oxygen), Q²=O (oxygen), R¹=2-fluoro-ethoxy, R³=dimethylamino.

In this case, R² has, for example, the meanings given above in group 1.

Group 59

n=2, Q¹=O (oxygen), Q²=O (oxygen), R¹=trifluoromethoxy, R³=dimethylamino.

In this case, R² has, for example, the meanings given above in group 1.

Group 60

n=2, Q¹=O (oxygen), Q²=O (oxygen), R¹=methoxycarbonyl, R³=dimethylamino.

In this case, R² has, for example, the meanings given above in group 1.

Group 61

n=2, Q¹=O (oxygen), Q²=O (oxygen), R¹=ethyl, R³=cyclopropyl.

In this case, R² has, for example, the meanings given above in group 1.

Group 62

n=2, Q¹=O (oxygen), Q²=O (oxygen), R¹=n-propyl, R³=cyclopropyl.

In this case, R² has, for example, the meanings given above in group 1.

Group 63

n=2, Q¹=O (oxygen), Q²=O (oxygen), R¹=i-propyl, R³=cyclopropyl.

In this case, R² has, for example, the meanings given above in group 1.

Group 64

n=2, Q¹=O (oxygen), Q²=O (oxygen), R¹=methoxy, R³=cyclopropyl.

In this case, R² has, for example, the meanings given above in group 1.

Group 65

n=2, Q¹=O (oxygen), Q²=(oxygen), R¹=ethoxy, R³=cyclopropyl.

In this case, R² has, for example, the meanings given above in group 1.

Group 66

n=2, Q¹=O (oxygen), Q²=O (oxygen), R¹=n-propoxy, R³=cyclopropyl.

In this case, R² has, for example, the meanings given above in group 1.

Group 67

n=2, Q¹=O (oxygen), Q²=O (oxygen), R¹=i-propoxy, R³=cyclopropyl.

In this case, R² has, for example, the meanings given above in group 1.

Group 68

n=2, Q¹=O (oxygen), Q²=O (oxygen), R¹=2-fluoro-ethoxy, R³=cyclopropyl.

In this case, R² has, for example, the meanings given above in group 1.

Group 69

n=2, Q¹=O (oxygen), Q²=O (oxygen), R¹=trifluoromethoxy, R³=cyclopropyl.

In this case, R² has, for example, the meanings given above in group 1.

Group 70

n=2, Q¹=O (oxygen), Q²=O (oxygen), R¹=methoxycarbonyl, R³=cyclopropyl.

In this case, R² has, for example, the meanings given above in group 1.

Group 71

n=2, Q¹=O (oxygen), Q²=O (oxygen), R¹=ethyl, R³=cyclopropylmethyl.

In this case, R² has, for example, the meanings given above in group 1.

Group 72

n=2, Q¹=O (oxygen), Q²=O (oxygen), R¹=n-propyl, R³=cyclopropylmethyl.

In this case, R² has, for example, the meanings given above in group 1.

Group 73

n=2, Q¹=O (oxygen), Q²=O (oxygen), R¹=i-propyl, R³=cyclopropylmethyl.

In this case, R² has, for example, the meanings given above in group 1.

Group 74

n=2, Q¹=O (oxygen), Q²=O (oxygen), R¹=methoxy, R³=cyclopropylmethyl.

In this case, R² has, for example, the meanings given above in group 1.

Group 75

n=2, Q¹=O (oxygen), Q²=O (oxygen), R¹=ethoxy, R³=cyclopropylmethyl.

In this case, R² has, for example, the meanings given above in group 1.

Group 76

n=2, Q¹=O (oxygen), Q²=O (oxygen), R¹=n-propoxy, R³=cyclopropylmethyl.

In this case, R² has, for example, the meanings given above in group 1.

Group 77

n=2, Q¹=O (oxygen), Q²=O (oxygen), R¹=i-propoxy, R³=cyclopropylmethyl.

In this case, R² has, for example, the meanings given above in group 1.

Group 78

n=2, Q¹=O (oxygen), Q²=O (oxygen), R¹=2-fluoro-ethoxy, R³=cyclopropylmethyl.

In this case, R² has, for example, the meanings given above in group 1.

Group 79

n=2, Q¹=O (oxygen), Q²=O (oxygen), R¹=trifluoromethoxy, R³=cyclopropylmethyl.

In this case, R² has, for example, the meanings given above in group 1.

Group 80

n=2, Q¹=O (oxygen), Q²=O (oxygen), R¹=methoxycarbonyl, R³=cyclopropylmethyl.

In this case, R² has, for example, the meanings given above in group 1.

Group 81

n=3, Q¹=O (oxygen), Q²=O (oxygen), R¹=methyl, R³=methyl.

In this case, R² has, for example, the meanings given above in group 1.

Group 82

n=3, Q¹=O (oxygen), Q²=O (oxygen), R¹=ethyl, R³=methyl.

In this case, R² has, for example, the meanings given above in group 1.

Group 83

n=3, Q¹=O (oxygen), Q²=O (oxygen), R¹=n-propyl, R³=methyl.

In this case, R² has, for example, the meanings given above in group 1.

Group 84

n=3, Q¹=O (oxygen), Q²=O (oxygen), R¹=i-propyl, R³=methyl.

In this case, R² has, for example, the meanings given above in group 1.

Group 85

n=3, Q¹=O (oxygen), Q²=O (oxygen), R¹=methoxy, R³=methyl.

In this case, R² has, for example, the meanings given above in group 1.

Group 86

n=3, Q¹=O (oxygen), Q²=O (oxygen), R¹=ethoxy, R³=methyl.

In this case, R² has, for example, the meanings given above in group 1.

Group 87

n=3, Q¹=O (oxygen), Q²=O (oxygen), R¹=n-propoxy, R³=methyl.

In this case, R² has, for example, the meanings given above in group 1.

Group 88

n=3, Q¹=O (oxygen), Q²=O (oxygen), R¹=i-propoxy, R³=methyl.

In this case, R² has, for example, the meanings given above in group 1.

Group 89

n=3, Q¹=O (oxygen), Q²=O (oxygen), R¹=2-fluoro-ethoxy, R³=methyl.

In this case, R² has, for example, the meanings given above in group 1.

Group 90

n=3, Q¹=O (oxygen), Q²=O (oxygen), R¹=trifluoromethoxy, R³=methyl.

In this case, R² has, for example, the meanings given above in group 1.

Group 91

n=3, Q¹=O (oxygen), Q²=O (oxygen), R¹=methoxycarbonyl, R³=methyl.

In this case, R² has, for example, the meanings given above in group 1.

Group 92

n=3, Q¹=O (oxygen), Q²=O (oxygen), R¹=methyl, R³=ethyl.

In this case, R² has, for example, the meanings given above in group 1.

Group 93

n=3, Q¹=O (oxygen), Q²=O (oxygen), R¹=ethyl, R³=ethyl.

In this case, R² has, for example, the meanings given above in group 1.

Group 94

n=3, Q¹=O (oxygen), Q²=O (oxygen), R¹=n-propyl, R³=ethyl.

In this case, R² has, for example, the meanings given above in group 1.

Group 95

n=3, Q¹=O (oxygen), Q²=O (oxygen), R¹=i-propyl, R³=ethyl.

In this case, R² has, for example, the meanings given above in group 1.

Group 96

n=3, Q¹=O (oxygen), Q²=O (oxygen), R¹=methoxy, R³=ethyl.

In this case, R² has, for example, the meanings given above in group 1.

Group 97

n=3, Q¹=O (oxygen), Q²=O (oxygen), R¹=ethoxy, R³=ethyl.

In this case, R² has, for example, the meanings given above in group 1.

Group 98

n=3, Q¹=O (oxygen), Q²=O (oxygen), R¹=n-propoxy, R³=ethyl.

In this case, R² has, for example, the meanings given above in group 1.

Group 99

n=3, Q¹=O (oxygen), Q²=O (oxygen), R¹=i-propoxy, R³=ethyl.

In this case, R² has, for example, the meanings given above in group 1.

Group 100

n=3, Q¹=O (oxygen), Q²=O (oxygen), R¹=2-fluoro-ethoxy, R³=ethyl.

In this case, R² has, for example, the meanings given above in group 1.

Group 101

n=3, Q¹=O (oxygen), Q²=O (oxygen), R¹=trifluoromethoxy, R³=ethyl.

In this case, R² has, for example, the meanings given above in group 1.

Group 102

n=3, Q¹=O (oxygen), Q²=O (oxygen), R¹=methoxycarbonyl, R³=ethyl.

In this case, R² has, for example, the meanings given above in group 1.

Group 103

n=3, Q¹=O (oxygen), Q²=O (oxygen), R¹=methyl, R³=methoxy.

In this case, R² has, for example, the meanings given above in group 1.

Group 104

n=3, Q¹=O (oxygen), Q²=O (oxygen), R¹=ethyl, R³=methoxy.

In this case, R² has, for example, the meanings given above in group 1.

Group 105

n=3, Q¹=O (oxygen), Q²=O (oxygen), R¹=n-propyl, R³=methoxy.

In this case, R² has, for example, the meanings given above in group 1.

Group 106

n=3, Q¹=O (oxygen), Q²=O (oxygen), R¹=i-propyl, R³=methoxy.

In this case, R² has, for example, the meanings given above in group 1.

Group 107

n=3, Q¹=O (oxygen), Q²=O (oxygen), R¹=methoxy, R³=methoxy.

In this case, R² has, for example, the meanings given above in group 1.

Group 108

n=3, Q¹=O (oxygen), Q²=O (oxygen), R¹=ethoxy, R³=methoxy.

In this case, R² has, for example, the meanings given above in group 1.

Group 109

n=3, Q¹=O (oxygen), Q²=O (oxygen), R¹=n-propoxy, R³=methoxy.

In this case, R² has, for example, the meanings given above in group 1.

Group 110

n=3, Q¹=O (oxygen), Q²=O (oxygen), R¹=i-propoxy, R³=methoxy.

In this case, R² has, for example, the meanings given above in group 1.

Group 111

n=3, Q¹=O (oxygen), Q²=O (oxygen), R¹=2-fluoro-ethoxy, R³=methoxy.

In this case, R² has, for example, the meanings given above in group 1.

Group 112

n=3, Q¹=O (oxygen), Q²=O (oxygen), R¹=trifluoromethoxy, R³=methoxy.

In this case, R² has, for example, the meanings given above in group 1.

Group 113

n=3, Q¹=O (oxygen), Q²=O (oxygen), R¹=methoxycarbonyl, R³=methoxy.

In this case, R² has, for example, the meanings given above in group 1.

Group 114

n=3, Q¹=O (oxygen), Q²=O (oxygen), R¹=methyl, R³=ethoxy.

In this case, R² has, for example, the meanings given above in group 1.

Group 115

n=3, Q¹=O (oxygen), Q²=O (oxygen), R¹=ethyl, R³=ethoxy.

In this case, R² has, for example, the meanings given above in group 1.

Group 116

n=3, Q¹=O (oxygen), Q²=O (oxygen), R¹=n-propyl, R³=ethoxy.

In this case, R² has, for example, the meanings given above in group 1.

Group 117

n=3, Q¹=O (oxygen), Q²=O (oxygen), R¹=i-propyl, R³=ethoxy.

In this case, R² has, for example, the meanings given above in group 1.

Group 118

n=3, Q¹=O (oxygen), Q²=O (oxygen), R¹=methoxy, R³=ethoxy.

In this case, R² has, for example, the meanings given above in group 1.

Group 119

n=3, Q¹=O (oxygen), Q²=O (oxygen), R¹=ethoxy, R³=ethoxy.

In this case, R² has, for example, the meanings given above in group 1.

Group 120

n=3, Q¹=O (oxygen), Q²=O (oxygen), R¹=n-propoxy, R³=ethoxy.

In this case, R² has, for example, the meanings given above in group 1.

Group 121

n=3, Q¹=O (oxygen), Q²=O (oxygen), R¹=i-propoxy, R³=ethoxy.

In this case, R² has, for example, the meanings given above in group 1.

Group 122

n=3, Q¹=O (oxygen), Q²=O (oxygen), R¹=2-fluoro-ethoxy, R³=ethoxy.

In this case, R² has, for example, the meanings given above in group 1.

Group 123

n=3, Q¹=O (oxygen), Q²=O (oxygen), R¹=trifluoromethoxy, R³=ethoxy.

In this case, R² has, for example, the meanings given above in group 1.

Group 124

n=3, Q¹=O (oxygen), Q²=O (oxygen), R¹=methoxycarbonyl, R³=ethoxy.

In this case, R² has, for example, the meanings given above in group 1.

Group 125

n=3, Q¹=O (oxygen), Q²=O (oxygen), R¹=methyl, R³=propargyloxy.

In this case, R² has, for example, the meanings given above in group 1.

Group 126

n=3, Q¹=O (oxygen), Q²=O (oxygen), R¹=ethyl, R³=propargyloxy.

In this case, R² has, for example, the meanings given above in group 1.

Group 127

n=3, Q¹=O (oxygen), Q²=O (oxygen), R¹=n-propyl, R³=propargyloxy.

In this case, R² has, for example, the meanings given above in group 1.

Group 128

n=3, Q¹=O (oxygen), Q²=O (oxygen), R¹=i-propyl, R³=propargyloxy.

In this case, R² has, for example, the meanings given above in group 1.

Group 129

n=3, Q¹=O (oxygen), Q²=O (oxygen), R¹=methoxy, R³=propargyloxy.

In this case, R² has, for example, the meanings given above in group 1.

Group 130

n=3, Q¹=O (oxygen), Q²=O (oxygen), R¹=ethoxy, R³=propargyloxy.

In this case, R² has, for example, the meanings given above in group 1.

Group 131

n=3, Q¹=O (oxygen), Q²=O (oxygen), R¹=n-propoxy, R³=propargyloxy.

In this case, R² has, for example, the meanings given above in group 1.

Group 132

n=3, Q¹=O (oxygen), Q²=O (oxygen), R¹=i-propoxy, R³=propargyloxy.

In this case, R² has, for example, the meanings given above in group 1.

Group 133

n=3, Q¹=O (oxygen), Q²=O (oxygen), R¹=2-fluoro-ethoxy, R³=propargyloxy.

In this case, R² has, for example, the meanings given above in group 1.

Group 134

n=3, Q¹=O (oxygen), Q²=O (oxygen), R¹=trifluoromethoxy, R³=propargyloxy.

In this case, R² has, for example, the meanings given above in group 1.

Group 135

n=3, Q¹=O (oxygen), Q²=O (oxygen), R¹=methoxycarbonyl, R³=propargyloxy.

In this case, R² has, for example, the meanings given above in group 1.

Group 136

n=3, Q¹=O (oxygen), Q²=O (oxygen), R¹=methyl, R=dimethylamino.

In this case, R² has, for example, the meanings given above in group 1.

Group 136

n=3, Q¹=O (oxygen), Q²=O (oxygen), R¹=ethyl, R³=dimethylamino.

In this case, R² has, for example, the meanings given above in group 1.

Group 137

n=3, Q¹=O (oxygen), Q²=O (oxygen), R¹=n-propyl, R³=dimethylamino.

In this case, R² has, for example, the meanings given above in group 1.

Group 138

n=3, Q¹=O (oxygen), Q²=O (oxygen), R¹=i-propyl, R³=dimethylamino.

In this case, R² has, for example, the meanings given above in group 1.

Group 139

n=3, Q¹=O (oxygen), Q²=O (oxygen), R¹=methoxy, R³=dimethylamino.

In this case, R² has, for example, the meanings given above in group 1.

Group 140

n=3, Q¹=O (oxygen), Q²=O (oxygen), R¹=ethoxy, R³=dimethylamino.

In this case, R² has, for example, the meanings given above in group 1.

Group 141

n=3, Q¹=O (oxygen), Q²=O (oxygen), R¹=n-propoxy, R³=dimethylamino.

In this case, R² has, for example, the meanings given above in group 1.

Group 142

n=3, Q¹=O (oxygen), Q²=O (oxygen), R¹=i-propoxy, R³=dimethylamino.

In this case, R² has, for example, the meanings given above in group 1.

Group 143

n=3, Q¹=O (oxygen), Q²=O (oxygen), R¹=2-fluoro-ethoxy, R³=dimethylamino.

In this case, R² has, for example, the meanings given above in group 1.

Group 144

n=3, Q¹=O (oxygen), Q²=O (oxygen), R¹=trifluoromethoxy, R³=dimethylamino.

In this case, R² has, for example, the meanings given above in group 1.

Group 145

n=3, Q¹=O (oxygen), Q²=O (oxygen), R¹=methoxycarbonyl, R³=dimethylamino.

In this case, R² has, for example, the meanings given above in group 1.

Group 146

n=2, Q¹=O (oxygen), Q²=O (oxygen), R¹=methyl, R³=cyclopropyl.

In this case, R² has, for example, the meanings given above in group 1.

Group 147

n=2, Q¹=O (oxygen), Q²=O (oxygen), R¹=ethyl, R³=cyclopropyl.

In this case, R² has, for example, the meanings given above in group 1.

Group 148

n=3, Q¹=O (oxygen), Q²=O (oxygen), R¹=n-propyl, R³=cyclopropyl.

In this case, R² has, for example, the meanings given above in group 1.

Group 149

n=3, Q¹=O (oxygen), Q²=O (oxygen), R¹=i-propyl, R³=cyclopropyl.

In this case, R² has, for example, the meanings given above in group 1.

Group 150

n=3, Q¹=O (oxygen), Q²=O (oxygen), R¹=methoxy, R³=cyclopropyl.

In this case, R² has, for example, the meanings given above in group 1.

Group 151

n=3, Q¹=O (oxygen), Q²=O (oxygen), R¹=ethoxy, R³=cyclopropyl.

In this case, R² has, for example, the meanings given above in group 1.

Group 152

n=3, Q¹=O (oxygen), Q²=O (oxygen), R¹=n-propoxy, R³=cyclopropyl.

In this case, R² has, for example, the meanings given above in group 1.

Group 153

n=3, Q¹=O (oxygen), Q²=O (oxygen), R¹=i-propoxy, R³=cyclopropyl.

In this case, R² has, for example, the meanings given above in group 1.

Group 154

n=3, Q¹=O (oxygen), Q²=O (oxygen), R¹=2-fluoro-ethoxy, R³=cyclopropyl.

In this case, R² has, for example, the meanings given above in group 1.

Group 155

n=3, Q¹=O (oxygen), Q²=O (oxygen), R¹=trifluoromethoxy, R³=cyclopropyl.

In this case, R² has, for example, the meanings given above in group 1.

Group 156

n=3, Q¹=O (oxygen), Q²=O (oxygen), R¹=methoxycarbonyl, R³=cyclopropyl.

In this case, R² has, for example, the meanings given above in group 1.

Group 157

n=3, Q¹=O (oxygen), Q²=O (oxygen), R¹=methyl, R³=cyclopropylmethyl.

In this case, R² has, for example, the meanings given above in group 1.

Group 158

n=3, Q¹=O (oxygen), Q²=O (oxygen), R¹=ethyl, R³=cyclopropylmethyl.

In this case, R² has, for example, the meanings given above in group 1.

Group 159

n=3, Q¹=O (oxygen), Q²=O (oxygen), R¹=n-propyl, R³=cyclopropylmethyl.

In this case, R² has, for example, the meanings given above in group 1.

Group 160

n=3, Q¹=O (oxygen), Q²=O (oxygen), R¹=i-propyl, R³=cyclopropylmethyl.

In this case, R² has, for example, the meanings given above in group 1.

Group 161

n=3, Q¹=O (oxygen), Q²=O (oxygen), R¹=methoxy, R³=cyclopropylmethyl.

In this case, R² has, for example, the meanings given above in group 1.

Group 162

n=3, Q¹=O (oxygen), Q²=O (oxygen), R¹=ethoxy, R³=cyclopropylmethyl.

In this case, R² has, for example, the meanings given above in group 1.

Group 163

n=3, Q¹=O (oxygen), Q²=O (oxygen), R¹=n-propoxy, R³=cyclopropylmethyl.

In this case, R² has, for example, the meanings given above in group 1.

Group 164

n=3, Q¹=O (oxygen), Q²=O (oxygen), R¹=i-propoxy, R³=cyclopropylmethyl.

In this case, R² has, for example, the meanings given above in group 1.

Group 165

n=3, Q¹=O (oxygen), Q²=O (oxygen), R¹=2-fluoro-ethoxy, R³=cyclopropylmethyl.

In this case, R² has, for example, the meanings given above in group 1.

Group 166

n=3, Q¹=O (oxygen), Q²=O (oxygen), R¹=trifluoromethoxy, R³=cyclopropylmethyl.

In this case, R² has, for example, the meanings given above in group 1.

Group 167

n=3, Q¹=O (oxygen), Q²=O (oxygen), R¹=methoxycarbonyl, R³=cyclopropylmethyl.

In this case, R² has, for example, the meanings given above in group 1.

A particular group of compounds of the formula (I) which may be mentioned are the compounds in which R¹ does not represent methyl if n represents 2.

The novel substituted fluoroalkoxyphenylsulphonylamino(thio)carbonyl-triazolin(ethi)ones of the general formula (I) have strong and selective herbicidal activity.

The novel substituted fluoroalkoxyphenylsulphonylamino(thio)carbonyl-triazolin(ethi)ones of the general formula (I) are obtained when

(a) fluoroalkoxybenzenesulphonamide of the general formula (II)

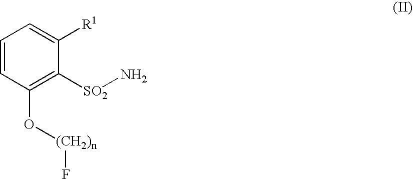

in which

n and R¹ are as defined above,

are reacted with substituted (thio)carbonyl-triazolin(ethi)ones of the general formula (III)

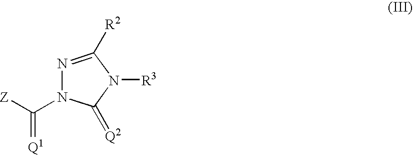

in which

Q¹, Q², R² and R³ are as defined above and

Z represents halogen or represents in each case optionally substituted alkoxy, aryloxy or arylalkoxy,

if appropriate in the presence of one or more reaction auxiliaries and if appropriate in the presence of one or more diluents, or when

(b) fluoroalkoxyphenylsulphonyl iso(thio)cyanates of the general formula (IV)

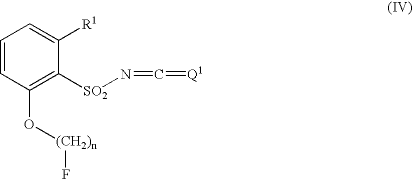

in which

n, Q¹ and R¹ are as defined above,

are reacted with triazolin(ethi)ones of the general formula (V)

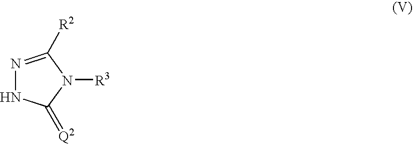

in which

Q², R² and R³ are as defined above,

if appropriate in the presence of one or more reaction auxiliaries and if appropriate in the presence of one or more diluents,

or when

(c) fluoroalkoxybenzenesulphonyl chlorides of the general formula (VI)

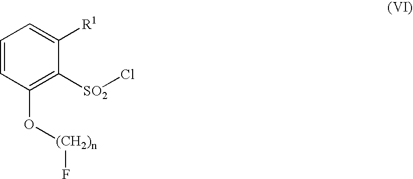

in which

n and R¹ are as defined above,

are reacted with triazolin(ethi)ones of the general formula (V)

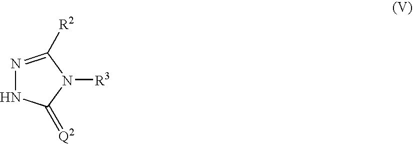

in which

Q², R² and R³ are as defined above,

and metal (thio)cyanates of the general formula (VII)

MQ¹CN (VII)

in which

Q¹ is as defined above and

M represents a metal equivalent,

if appropriate in the presence of one or more reaction auxiliaries and if appropriate in the presence of one or more diluents,

and the compounds of the general formula (I) obtained by process (a), (b) or (c) are, if appropriate, converted into salts using customary methods.

Using, for example, 2-(2-fluoro-ethoxy)-6-methoxy-benzenesulphonamide and 5-ethoxy-4-methyl-2-phenoxycarbonyl-2,4-dihydro-3H-1,2,4-triazole-3-thione as starting materials, the course of the reaction of the process (a) according to the invention can be illustrated by the formula scheme below:

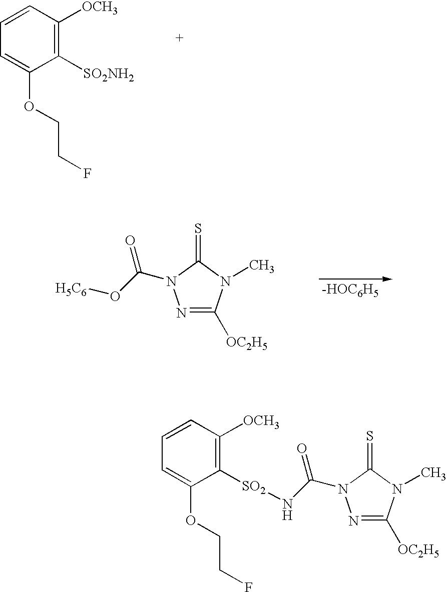

Using, for example, 2-(2-fluoro-ethoxy)-6-trifluoromethyl-phenylsulphonyl isothiocyanate and 5-ethyl-4-methoxy-2,4-dihydro-3H-1,2,4-triazol-3-one as starting materials, the course of the reaction of the process (b) according to the invention can be illustrated by the following formula scheme:

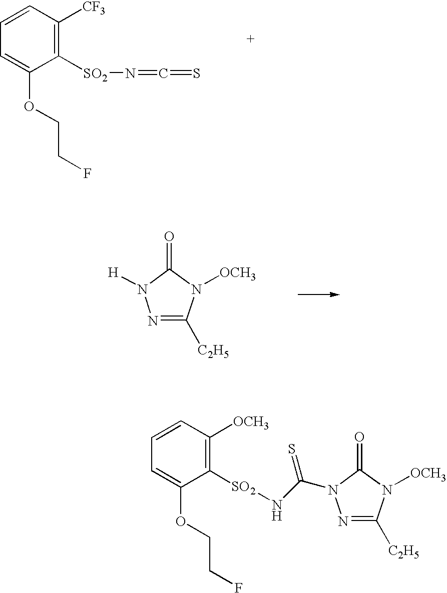

Using, for example, 2-(3-fluoro-propoxy)-6-propyl-benzenesulphonyl chloride, 5-ethylthio-4-methoxy-2,4-dihydro-3H-1,2,4-triazol-3-one and potassium cyanate as starting materials, the course of the reaction of the process (c) can be illustrated by the following formula scheme:

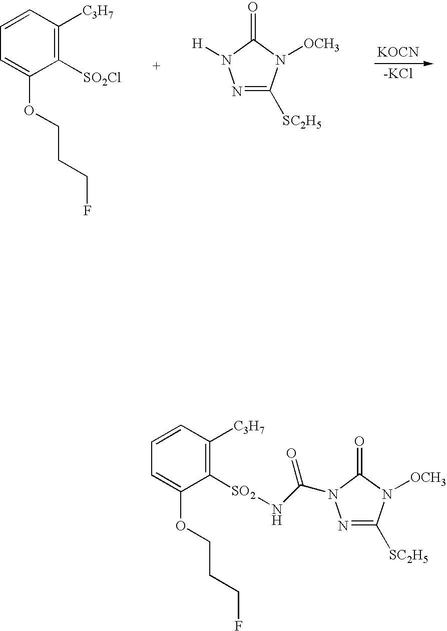

The formula (II) provides a general definition of the fluoroalkoxybenzenesulphonamides for use as starting materials in the process (a) according to the invention for preparing compounds of the general formula (I). In the general formula (II), n and R¹ preferably or in particular have those meanings which have already been mentioned above, in connection with the description of the compounds of the general formula (I) according to the invention, as being preferred or as being particularly preferred for n and R¹.

Some of the starting materials of the general formula (II) are known (cf. WO-A-97/03056). They are also the subject of an earlier, but not prior-published, application (cf. German patent application No. 10111649, submitted Dec. 3, 2001).

The fluoroalkoxybenzenesulphonamides of the general formula (II) are obtained when hydroxybenzenesulphonamides of the general formula (VIII)

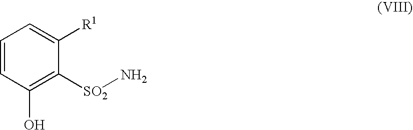

in which

R¹ is as defined above,

are reacted with ω-fluoro-α-halogeno-alkanes of the general formula (IX)

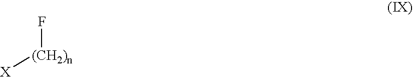

in which

n is as defined above and

X represents halogen, preferably chlorine, bromine or iodine, in particular bromine, or represents methylsulphonyloxy, phenylsulphonyloxy or tolylsulphonyloxy,

if appropriate in the presence of a diluent, such as, for example, acetone, butanone, acetonitrile, propionitrile, N,N-dimethyl-formamide or N,N-dimethyl-acetamide, and if appropriate in the presence of an acid acceptor, such as, for example, potassium carbonate, at temperatures between 0° C. and 150° C. (cf. the Preparation Examples).

The hydroxybenzenesulphonamides of the general formula (VIII) required as intermediates are known and/or can be prepared by processes known per se (cf. EP-A-44807, WO-A-97/03056).

The ω-fluoro-α-halogeno-alkanes of the general formula (IX) furthermore required as intermediates are known organic chemicals for synthesis.

The formula (III) provides a general definition of the (thio)carbonyltriazolin(ethi)ones further to be used as starting materials in the process (a) according to the invention for preparing compounds of the general formula (I). In the general formula (III), Q¹, Q², R² and R³ preferably or in particular have those meanings which already been mentioned above, in connection with the description of the compounds of the general formula (I) according to the invention, as being preferred or as being particularly preferred for Q¹, Q², R² and R³; Z preferably represents fluorine, chlorine, bromine, C₁-C₄-alkoxy, or represents in each case optionally nitro-, chlorine- or methyl-substituted phenoxy or benzyloxy, in particular chlorine, methoxy, ethoxy or phenoxy.

The starting materials of the general formula (III) are known and/or can be prepared by processes known per se (cf. EP-A-459244, EP-A-341489, EP-A-422469, EP-A-425948, EP-A-431291, EP-A-507171, EP-A-534266).

The formula (IV) provides a general definition of the fluoroalkoxyphenylsulphonyl iso(thio)cyanates to be used as starting materials in the process (b) according to the invention for preparing compounds of the general formula (I). In the general formula (IV), n, Q¹ and R¹ preferably or in particular have those meanings which have already been mentioned above, in connection with the description of the compounds of the general formula (I) according to the invention, as being preferred or as being particularly preferred for n, Q¹ and R¹.

The starting materials of the general formula (IV) have hitherto not been disclosed in the literature; as novel substances, they also form part of the subject-matter of the present application.

The novel fluoroalkoxyphenylsulphonyl iso(thio)cyanates of the general formula (IV) are obtained when fluoroalkoxybenzenesulphonamides of the general formula (II)

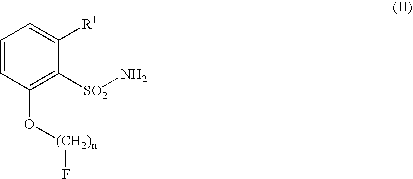

in which

n and R¹ are as defined above,

are reacted with phosgene or thiophosgene, if appropriate in the presence of an alkyl isocyanate, such as, for example, butyl isocyanate, if appropriate in the presence of a reaction auxiliary, such as, for example, diazabicyclo[2.2.2]octane, and in the presence of a diluent, such as, for example, toluene, xylene or chlorobenzene, at temperatures between 80° C. and 150° C., and the volatile components are distilled off under reduced pressure after the reaction has ended.

The formula (V) provides a general definition of the triazolin(ethi)ones to be used as starting materials in the process (b) and (c) according to the invention for preparing compounds of the general formula (I). In the general formula (V), Q², R² and R³ preferably or in particular have those meanings which have already been mentioned above, in connection with the description of the compounds of the general formula (I) according to the invention, as being preferred or as being particularly preferred for Q², R² and R³.

The starting materials of the formula (V) are known and/or can be prepared by processes known per se (cf. EP-A-341489, EP-A-422469, EP-A425948, EP-A-431291, EP-A-507171, EP-A-534266).

The formula (VI) provides a general definition of the fluoroalkoxybenzenesulphonyl chlorides to be used as starting materials in the process (c) according to the invention for preparing compounds of the general formula (I). In the general formula (VI), n and R¹ preferably or in particular have those meanings which have already been mentioned above, in connection with the description of the compounds of the general formula (I) according to the invention, as being preferred or as being particularly preferred for n and R¹.

The starting materials of the general formula (VI) have hitherto not been disclosed in the literature; as novel substances, they also form part of the subject-matter of the present application.

The novel fluoroalkoxybenzenesulphonyl chlorides of the general formula (VI) are obtained when aniline derivatives of the general formula (X)

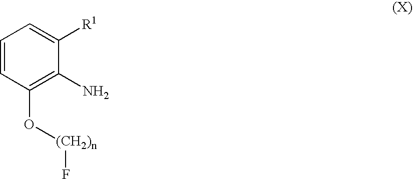

in which

n and R¹ are as defined above,

are reacted with an alkali metal nitrite, such as, for example, sodium nitrite, in the presence of hydrochloric acid at temperatures between −10° C. and +10° C., and the resulting diazonium salt solution is reacted with sulphur dioxide in the presence of a diluent, such as, for example, dichloromethane or 1,2-dichloro-ethane, and in the presence of a catalyst, such as, for example, copper(I) chloride, if appropriate in the presence of a further catalyst, such as, for example, dodecyltrimethylammonium bromide, at temperatures between −10° C. and +50° C.

The processes (a), (b) and (c) according to the invention are in each case preferably carried out in the presence of one or more reaction auxiliaries. Suitable reaction auxiliaries for the processes (a), (b) and (c) according to the invention are, in general the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or -i-propoxide, n-, i-, s- or -t-butoxide or potassium methoxide, ethoxide, n- or i-propoxide, n-, i-, s- or t-butoxide; furthermore also basic organic nitrogen compounds such as, for example, trimethylamine, triethylamine, tripropylamine, tributylamine, ethyl-diisopropyl amine, N,N-dimethyl-cyclohexyl amine, dicyclohexylamine, ethyl-dicyclohexylamine, N,N-dimethyl-aniline, N,N-dimethyl-benzylamine, pyridine, 2-methyl-, 3-methyl-, 4-methyl-, 2,4-dimethyl-, 2,6-dimethyl-, 3,4-dimethyl- and 3,5-dimethyl-pyridine, 5-ethyl-2-methyl-pyridine, 4-dimethylaminopyridine, N-methyl-piperidine, 1,4-diazabicyclo[2.2.2]-octane (DABCO), 1,5-diazabicyclo[4.3.0]-non-5-ene (DBN) or 1,8-diazabicyclo[5.4.0]-undec-7-ene (DBU).

Suitable further reaction auxiliaries for the processes (a), (b) and (c) are also phase-transfer catalysts. Examples of such catalysts which may be mentioned are:

Tetrabutylammonium bromide, tetrabutylammonium chloride, tetraoctylammonium chloride, tetrabutylammonium hydrogensulphate, methyl-trioctylammonium chloride, hexadecyl-trimethylammonium chloride, hexadecyl-trimethylammonium bromide, benzyl-trimethylammonium chloride, benzyl-triethylammonium chloride, benzyl-trimethylammonium hydroxide, benzyl-triethylammonium hydroxide, benzyl-tributylammonium chloride, benzyl-tributylammonium bromide, tetrabutylphosphonium bromide, tetrabutylphosphonium chloride, tributyl-hexadecylphosphonium bromide, butyl-triphenylphosphonium chloride, ethyl-trioctylphosphonium bromide, tetraphenylphosphonium bromide.

The processes (a), (b) and (c) according to the invention for preparing the compounds of the general formula (I) are in each case preferably carried out using one or more diluents. Suitable diluents for carrying out the processes (a), (b) and (c) according to the invention are, in addition to water, primarily inert organic solvents. These include, in particular, aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, dioxane, tetrahydrofuran or ethylene glycol dimethyl ether or ethylene glycol diethyl ether, ketones, such as acetone, butanone or methyl isobutyl ketone; nitriles, such as acetonitrile, propionitrile or butyronitrile; amides, such as N,N-dimethylformamide, N,N-dimethylacetamide, N-methyl-formanilide, N-methyl-pyrrolidone or hexamethylphosphoric triamide; esters, such as methyl acetate or ethyl acetate; sulphoxides, such as dimethyl sulphoxide; alcohols, such as methanol, ethanol, n- or i-propanol, ethylene glycol monomethyl ether, ethylene glycol monoethyl ether, diethylene glycol monomethyl ether, diethylene glycol monoethyl ether, mixtures thereof with water or pure water.

When carrying out the processes (a), (b) and (c) according to the invention, the reaction temperatures can in each case be varied within a relatively wide range. In general, the processes are carried out at temperatures between 0° C. and 150° C., preferably between 10° C. and 120° C.

The processes according to the invention are generally carried out under atmospheric pressure. However, it is also possible to carry out the processes according to the invention under elevated or reduced pressure—in general between 0.1 bar and 10 bar.

For carrying out the processes according to the invention, the starting materials are generally employed in approximately equimolar amounts. However, it is also possible to employ a relatively large excess of one of the components. The reaction is generally carried out in a suitable diluent in the presence of a reaction auxiliary and the reaction mixture is generally stirred at the required temperature for a number of hours. Work-up is carried out by customary methods (cf. the preparation examples). The active compounds according to the invention can be used as defoliants, desiccants, haulm killers and, especially, as weed killers. Weeds in the broadest sense are understood to mean all plants which grow in locations where they are undesired. Whether the substances according to the invention act as total or selective herbicides depends essentially on the amount used.

The active compounds according to the invention can be used, for example, in connection with the following plants:

Dicotyledonous weeds of the genera: Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

Dicotyledonous crops of the genera: Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia.

Monocotyledonous weeds of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Monocotyledonous crops of the genera: Allium, Ananas, Asparagus, Avena, Hordeum, Oryza, Panicum, Saccharum, Secale, Sorghum, Triticale, Triticum, Zea. However, the use of the active compounds according to the invention is in no way restricted to these genera, rather also extends in the same manner to other plants.

The active compounds according to the invention are suitable, depending on the concentration, for the total control of weeds, for example on industrial terrain and rail tracks, and on paths and areas with and without tree plantings. Similarly, the active compounds according to the invention can be employed for controlling weeds in perennial crops, for example forests, decorative tree plantings, orchards, vineyards, citrus groves, nut orchards, banana plantations, coffee plantations, tea plantations, rubber plantations, oil palm plantations, cocoa plantations, soft fruit plantings and hop fields, on lawns, turf and pastureland, and for the selective control of weeds in annual crops.

The compounds, of the formula (I) according to the invention have strong herbicidal activity and a broad active spectrum when used on the soil and on above-ground parts of plants. To a certain extent they are also suitable for the selective control of monocotyledonous and dicotyledonous weeds in monocotyledonous and dicotyledonous crops, both by the pre-emergence and by the post-emergence method.

At certain concentrations or application rates, the active compounds according to the invention can also be employed for controlling animal pests and fungal or bacterial plant diseases. If appropriate, they can also be used as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and including the cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

Treatment according to the invention of the plants and plant parts with the active compounds is carried out directly or by allowing the compounds to act on the surroundings, environment or storage space by the customary treatment methods, for example by immersion, spraying, evaporation, fogging, scattering, painting on and, in the case of propagation material, in particular in the case of seeds, also by applying one or more coats.

The active compounds can be converted into the customary formulations such as solutions, emulsions, wettable powders, suspensions, powders, dusts, pastes, soluble powders, granules, suspension-emulsion concentrates, natural and synthetic materials impregnated with active compound, and microencapsulations in polymeric materials.

These formulations are produced in a known manner, for example by mixing the active compounds with extenders, that is, liquid solvents and/or solid carriers, optionally with the use of surfactants, that is, emulsifiers and/or dispersants, and/or foam formers.

If the extender used is water, it is also possible, for example, to use organic solvents as cosolvents. The following are essentially suitable as liquid solvents: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water.

Suitable solid carriers are: for example ammonium salts and ground natural minerals such as kaolins, clays, talc, chalk, quartz, attapulgite, montmorillonite or diatomaceous earth, and ground synthetic materials such as highly-disperse silica, alumina and silicates; suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks; suitable emulsifiers and/or foam formers are: for example nonionic and anionic emulsifiers such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates; suitable dispersants are: for example lignin-sulphite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other additives can be mineral and vegetable oils.

It is possible to use colorants such as inorganic pigments, for example iron oxide, titanium oxide and Prussian Blue, and organic colorants such alizarin colorants, azo colorants and metal phthalocyanine colorants, and trace nutrients such as salts of iron, manganese, boron, copper, cobalt, molybdenum and zinc.

The formulations generally comprise between 0.1 and 95% by weight of active compound, preferably between 0.5 and 90%.

For controlling weeds, the active compounds according to the invention, as such or in their formulations, can also be used as mixtures with known herbicides and/or substances which improve the compatibility with crop plants (“safeners”), finished formulations or tank mixes being possible. Also possible are mixtures with weed-killers comprising one or more known herbicides and a safener.

Possible components for the mixtures are known herbicides, for example acetochlor, acifluorfen (-sodium), aclonifen, alachlor, alloxydim (-sodium), ametryne, amicarbazone, amidochlor, amidosulfuron, anilofos; asulam, atrazine, azafenidin, azimsulfuron, beflubutamid, benazolin (-ethyl), benfuresate, bensulfuron (-methyl), bentazon, benzfendizone, benzobicyclon, benzofenap, benzoylprop (-ethyl), bialaphos, bifenox, bispyribac (-sodium), bromobutide, bromofenoxim, bromoxynil, butachlor, butafenacil (-allyl), butroxydim, butylate, cafenstrole, caloxydim, carbetamide, carfentrazone (-ethyl), chlomethoxyfen, chloramben, chloridazon, chlorimuron (-ethyl), chlornitrofen, chlorsulfuron, chlortoluron, cinidon (-ethyl), cinmethylin, cinosulfuron, clefoxydim, clethodim, clodinafop (-propargyl), clomazone, clomeprop, clopyralid, clopyrasulfuron (-methyl), cloransulam (-methyl), cumyluron, cyanazine, cybutryne, cycloate, cyclosulfamuron, cycloxydim, cyhalofop (-butyl), 2,4-D, 2,4-DB, desmedipham, diallate, dicamba, dichlorprop (-P), diclofop (-methyl), diclosulam, diethatyl (-ethyl), difenzoquat, diflufenican, diflufenzopyr, dimefuron, dimepiperate, dimethachlor, dimetbametryn, dimethenamid, dimexyflam, dinitramine, diphenamid, diquat, dithiopyr, diuron, dymron, epropodan, EPTC, esprocarb, ethalfluralin, ethametsulfuron (-methyl), ethofumesate, ethoxyfen, ethoxysulfuron, etobenzanid, fenoxaprop (-P-ethyl), fentrazamide, flamprop (-isopropyl, -isopropyl-L, -methyl), flazasulfuron, florasulam, fluazifop (-P-butyl), fluazolate, flucarbazone (-sodium), flufenacet, flumetsulam, flumiclorac (-pentyl), flumioxazin, flumipropyn, flumetsulam, fluometuron, fluorochloridone, fluoroglycofen (-ethyl), flupoxam, flupropacil, flurpyrsulfuron (-methyl, -sodium), flurenol (-butyl), fluridone, fluroxypyr (-butoxypropyl, -meptyl), flurprimidol, flurtamone, fluthiacet (-methyl), fluthiamide, fomesafen, foramsulfuron, glufosinate (-ammonium), glyphosate (-isopropylammonium), halosafen, haloxyfop (-ethoxyethyl, -P-methyl), hexazinone, imazamethabenz (-methyl), imazamethapyr, imazamox, imazapic, imazapyr, imazaquin, imazethapyr, imazosulfuron, iodosulfuron (-methyl, -sodium), ioxynil, isopropalin, isoproturon, isouron, isoxaben, isoxachlortole, isoxaflutole, isoxapyrifop, lactofen, lenacil, linuron, MCPA, mecoprop, mefenacet, mesotrione, metamitron, metazachlor, methabenzthiazuron, metobenzuron, metobromuron, (alpha-) metolachlor, metosulam, metoxuron, metribuzin, metsulfuron (-methyl), molinate, monolinuron, naproanilide, napropamide, neburon, nicosulfuron, norflurazon, orbencarb, oryzalin, oxadiargyi, oxadiazon, oxasulfuron, oxaziclomefone, oxyfluorfen, paraquat, pelargonic acid, pendimethalin, pendralin, pentoxazone, phenmedipham, picolinafen, piperophos, pretilachlor, primisulfuron (-methyl), profluazol, prometryn, propachlor, propanil, propaquizafop, propisochlor, propoxycarbazone (-sodium), propyzamide, prosulfocarb, prosulfuron, pyraflufen (-ethyl), pyrazogyl, pyrazolate, pyrazosulfuron (-ethyl), pyrazoxyfen, pyribenzoxim, pyributicarb, pyridate, pyridatol, pyriftalid, pyriminobac (-methyl), pyrithiobac (-sodium), quinchlorac, quinmerac, quinoclamine, quizalofop(-P-ethyl, -P-tefuryl), rimsulfuron, sethoxydim, simazine, simetryn, sulcotrione, sulfentrazone, sulfometuron (-methyl), sulfosate, sulfosulfuron, tebutam, tebuthiuron, tepraloxydim, terbuthylazine, terbutryn, thenylchlor, thiafluamide, thiazopyr, thidiazimin, thifensulfuron (-methyl), thiobencarb, tiocarbazil, tralkoxydim, triallate, triasulfuron, tribenuron (-methyl), triclopyr, tridiphane, trifluralin, trifloxysulfuron, triflusulfuron (-methyl), tritosulfuron.

Also suitable for the mixtures are known safeners, for example

AD-67, BAS-145138, benoxacor, cloquintocet (-mexyl), cyometrinil, 2,4-D, DKA-24, dichlormid, dymron, fenclorim, fenchlorazol (-ethyl), flurazole, fluxofenim, furilazole, isoxadifen (-ethyl), MCPA, mecoprop (-P), mefenpyr (-diethyl), MG-191, oxabetrinil, PPG-1292, R-29148.

A mixture with other known active compounds, such as fungicides, insecticides, acaricides, nematicides, bird repellents, plant nutrients and agents which improve soil structure, is also possible.

The active compounds can be used as such, in the form of their formulations or in the use forms prepared therefrom by further dilution, such as ready-to-use solutions, suspensions, emulsions, powders, pastes and granules. They are used in a customary manner, for example by watering, spraying, atomizing or broadcasting.

The active compounds according to the invention can be applied both before and after emergence of the plants. They can also be incorporated into the soil before sowing.

The amount of active compound used can vary within a relatively wide range. It depends essentially on the nature of the desired effect. In general, the amounts used are between 1 g and 10 kg of active compound per hectare of soil surface, preferably between 5 g and 5 kg per ha.

As already mentioned above, it is possible to treat all plants and their parts according to the invention. In a preferred embodiment, wild plant species and cultivars, or those obtained by conventional biological breeding, such as crossing or protoplast fusion, and parts thereof, are treated. In a further preferred embodiment, transgenic plants and cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the cultivars which are in each case commercially available or in use are treated according to the invention. Cultivars are to be understood as meaning plants having certain properties (“traits”) and which have been obtained by conventional breeding, by mutagenesis or by recombinant DNA techniques. They can be varieties, bio- or genotypes.

Depending on the plant species or cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions—also in combination with the agrochemically active compounds—which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or cultivars (i.e. those obtained by genetic engineering) which are preferably treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are emphasized are in particular increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof) (hereinbelow referred to as “Bt plants”). Traits that are also particularly emphasized are the increased defence of the plants to fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylurea, for example maize). Herbicide-resistant plants (plants bred in a conventional manner for herbicide tolerance) which may be mentioned include the varieties sold under the name Clearfield® (for example maize). Of course, these statements also apply to cultivars having these genetic traits or genetic traits still to be developed, which plants will be developed and/or marketed in the future.

The plants listed can be treated according to the invention in a particularly advantageous manner with the compounds of the formula I or the mixtures of active compounds according to the invention where, in addition to the effective control of the weed plants, the abovementioned synergistic effects with the transgenic plants or plant cultivators occur. The preferred ranges stated above for the active compounds or mixtures also apply to the treatment of these plants. Particular emphasis is given to the treatment of plants with the mixtures specifically mentioned in the present text.

The preparation and the use of the active compounds according to the invention is illustrated by the examples below.

### PREPARATION EXAMPLES

### Example 1

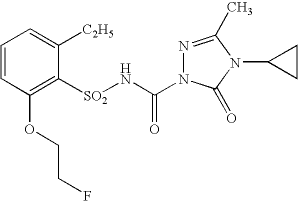

(Process (a))

A mixture of 1.8 g (7.0 mmol) of 2-(2-fluoro-ethoxy)-6-ethyl-benzenesulphonamide, 1.8 g (7.0 mmol) of 4-cyclopropyl-5-methyl-2-phenoxycarbonyl-2,4-dihydro-3H-1,2,4-triazol-3-one, 1.2 g (7.7 mmol) of 1,8-diazabicyclo[5.4.0]-undec-7-ene (DBU) and 20 ml of acetonitrile is stirred at room temperature (about. 20° C.) for 60 minutes and then diluted with approximately double the volume of methylene chloride and shaken with 2N hydrochloric acid. The organic phase is then dried with sodium sulphate and filtered. The filtrate is concentrated under reduced pressure, the residue is digested with diisopropyl ether and the resulting crystalline product is isolated by filtration with suction.

This gives 2.1 g (73% of theory) of 4-cyclopropyl-5-methyl-2-[(2-fluoro-ethoxy)-6-ethyl-phenylsulphonylaminocarbonyl]-2,4-dihydro-3H-1,2,4-triazol-3-one of melting point 147° C.

Analogously to Example 1, and in accordance with the general description of the preparation process according to the invention, it is also possible to prepare, for example, the compounds of the general formula (I) listed in Table 1 below.

TABLE 1
Examples of the compounds of the formula (I)

| | |
|---|---|
| | (I) |
| 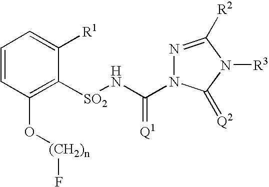 | |

| Ex. | | | | | | | Melting point |
|---|---|---|---|---|---|---|---|
| No. | n | Q¹ | Q² | R¹ | R² | R³ | (° C.) |
| 2 | 2 | O | O | C₂H₅ | C₂H₅ | OC₂H₅ | 118 |
| 3 | 2 | O | O | C₂H₅ | OCH₃ | CH₃ | 166 |
| 4 | 2 | O | O | C₂H₅ | N(CH₃)₂ | CH₃ | 170 |
| 5 | 2 | O | O | C₂H₅ | SCH₃ | CH₃ | 168 |
| 6 | 2 | O | O | C₂H₅ | OCH₃ | 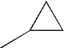 | 156 |
| 7 | 2 | O | O | C₂H₅ | CH₃ | CH₃ | 290 |
| | | | | | | | Na salt |
| 8 | 2 | O | O | C₂H₅ | C₃H₇-n | CH₃ | 274 |
| | | | | | | | Na salt |
| 9 | 2 | O | O | C₂H₅ | OC₂H₅ | CH₃ | 140 |
| 10 | 2 | O | O | C₂H₅ | OC₃H₇-n | CH₃ | 138 |
| 11 | 2 | O | O | C₂H₅ | OC₃H₇-i | CH₃ | 136 |
| 12 | 2 | O | O | C₂H₅ | 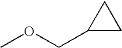 | CH₃ | 139 |
| 13 | 2 | O | O | C₂H₅ | OC₂H₅ | 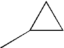 | 138 |
| 14 | 2 | O | O | C₂H₅ | OC₃H₇-n | 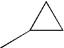 | 125 |
| 15 | 2 | O | O | C₂H₅ | OC₃H₇-i | 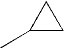 | 140 |
| 16 | 2 | O | O | C₂H₅ | 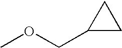 | 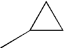 | 158 |
| 17 | 2 | O | O | C₂H₅ | 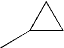 | CH₃ | 157 |
| 18 | 2 | O | O | C₂H₅ | CH₂OCH₃ | CH₃ | 101 |
| 19 | 2 | O | O | C₃H₇-n | CH₃ | CH₃ | 290 |
| | | | | | | | Na salt |
| 20 | 2 | O | O | C₃H₇-n | C₃H₇-n | CH₃ | 108 |
| 21 | 2 | O | O | C₃H₇-n | OCH₃ | CH₃ | 158 |
| 22 | 2 | O | O | C₃H₇-n | OC₂H₅ | CH₃ | 152 |
| 23 | 2 | O | O | C₃H₇-n | OC₃H₇-i | CH₃ | 164 |
| 24 | 2 | O | O | C₃H₇-n | OCH₃ | 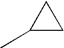 | 137 |
| 25 | 2 | O | O | C₃H₇-n | OC₂H₅ | 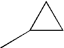 | 117 |
| 26 | 2 | O | O | C₃H₇-n | OC₃H₇-i | 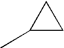 | 158 |
| 27 | 2 | O | O | C₃H₇-n | N(CH₃)₂ | CH₃ | 147 |
| 28 | 2 | O | O | C₃H₇-n | C₂H₅ | OC₂H₅ | 237 |
| | | | | | | | Na salt |
| 29 | 2 | O | O | C₃H₇-n | SCH₃ | CH₃ | 130 |
| | | | | | | | Na salt |
| 30 | 2 | O | O | C₃H₇-i | CH₃ | CH₃ | 286 |
| | | | | | | | Na salt |
| 31 | 2 | O | O | C₃H₇-i | CH₃ | CH₃ | 167 |
| 32 | 2 | O | O | OC₃H₇-n | OC₂H₅ | CH₃ | 132 |
| 33 | 2 | O | O | OC₂H₅ | OC₂H₅ | CH₃ | 157 |
| 34 | 2 | O | O | OC₂H₅ | OC₃H₇-n | CH₃ | 123 |
| 35 | 2 | O | O | OC₂H₅ | SCH₃ | CH₃ | 118 |
| 36 | 3 | O | O | CH₃ | OCH₂CF₃ | CH₃ | 107 |
| 37 | 3 | O | O | CH₃ | OCH₂CF₃ | 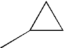 | 114 |
| 38 | 3 | O | O | CH₃ | CH₃ | CH₃ | 105 |
| 39 | 3 | O | O | CH₃ | C₄H₉-i | CH₃ | 105 |
| 40 | 3 | O | O | CH₃ | CH₃ | 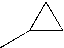 | 93 |
| 41 | 3 | O | O | CH₃ | OCH₃ | CH₃ | 120 |
| 42 | 3 | O | O | CH₃ | SCH₃ | CH₃ | 130 |
| 43 | 3 | O | O | CH₃ | OC₃H₇-n | CH₃ | 104 |
| 44 | 3 | O | O | CH₃ | OC₃H₇-n | 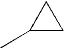 | 107 |
| 45 | 3 | O | O | CH₃ | OC₃H₇-i | 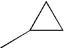 | 105 |
| 46 | 3 | O | O | CH₃ | C₃H₇-i | CH₃ | 131 |
| 47 | 3 | O | O | CH₃ | C₂H₅ | CH₃ | 87 |
| 48 | 3 | O | O | CH₃ | C₃H₇-n | CH₃ | 120 |
| 49 | 3 | O | O | CH₃ | OC₃H₇-i | CH₃ | 109 |
| 50 | 3 | O | O | CH₃ | OC₂H₅ | CH₃ | 120 |
| 51 | 3 | O | O | CH₃ | OCH₃ | 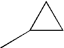 | 105 |
| 52 | 3 | O | O | CH₃ | Br | 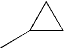 | 110 |
| 53 | 3 | O | O | CH₃ | Br | CH₃ | 115 |
| 54 | 3 | O | O | CH₃ | C₃H₇-n | OCH₃ | 92 |
| 55 | 3 | O | O | CH₃ | 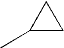 | 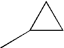 | 145 |
| 56 | 3 | O | O | CH₃ | C₂H₅ | OC₂H₅ | 95 |
| 57 | 3 | O | O | CH₃ | SCH₃ | 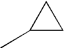 | 102 |
| 58 | 3 | O | O | CH₃ | CH₃ | 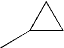 | 141 |
| 59 | 3 | O | O | CH₃ | SC₂H₅ | CH₃ | 118 |
| 60 | 3 | O | O | CH₃ | N(CH₃)₂ | CH₃ | 135 |
| 61 | 3 | O | O | CH₃ | C₂H₅ | 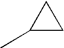 | 111 |
| 62 | 3 | O | O | CH₃ | C₃H₇-n | 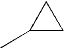 | 107 |
| 63 | 3 | O | O | CH₃ | C₃H₇-i | 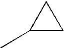 | 133 |
| 64 | 3 | O | O | CH₃ | SC₂H₅ | 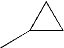 | 109 |
| 65 | 3 | O | O | CH₃ | N(CH₃)₂ | 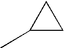 | 114 |
| 66 | 3 | O | O | CH₃ | OCH₃ | CH₃ | 230 |
| | | | | | | | Na salt |
| 67 | 3 | O | O | CH₃ | OC₂H₅ | CH₃ | 192 |
| | | | | | | | Na salt |
| 68 | 3 | O | O | CH₃ | CH₃ | CH₃ | 260 |
| | | | | | | | Na salt |
| 69 | 3 | O | O | C₂H₅ | OCH₂CF₃ | CH₃ | 106 |
| 70 | 3 | O | O | C₂H₅ | CH₃ | CH₃ | 92 |
| 71 | 3 | O | O | C₂H₅ | C₄H₉-i | CH₃ | 146 |
| 72 | 3 | O | O | C₂H₅ | CH₃ | 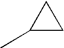 | 143 |
| 73 | 3 | O | O | C₂H₅ | OCH₃ | CH₃ | 115 |
| 74 | 3 | O | O | C₂H₅ | SCH₃ | CH₃ | 116 |
| 75 | 3 | O | O | C₂H₅ | OC₃H₇-n | CH₃ | 108 |
| 76 | 3 | O | O | C₂H₅ | OC₃H₇-n | 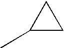 | 113 |
| 77 | 3 | O | O | C₂H₅ | OC₃H₇-i | 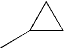 | 130 |
| 78 | 3 | O | O | C₂H₅ | C₃H₇-i | CH₃ | 100 |
| 79 | 3 | O | O | C₂H₅ | C₂H₅ | CH₃ | 98 |
| 80 | 3 | O | O | C₂H₅ | C₃H₇-n | CH₃ | 105 |
| 81 | 3 | O | O | C₂H₅ | OC₃H₇-i | CH₃ | 120 |
| 82 | 3 | O | O | C₂H₅ | OC₂H₅ | 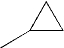 | 137 |
| 83 | 3 | O | O | C₂H₅ | OC₂H₅ | CH₃ | 122 |
| 84 | 3 | O | O | C₂H₅ | OCH₃ | 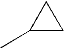 | 124 |
| 85 | 3 | O | O | C₂H₅ | Br | 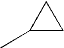 | 132 |
| 86 | 3 | O | O | C₂H₅ | Br | CH₃ | 162 |
| 87 | 3 | O | O | C₂H₅ | 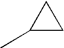 | 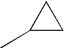 | 109 |
| 88 | 3 | O | O | C₂H₅ | C₂H₅ | OC₂H₅ | 104 |
| 89 | 3 | O | O | C₂H₅ | SCH₃ | 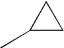 | 119 |
| 90 | 3 | O | O | C₂H₅ | CH₃ | 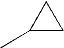 | 133 |
| 91 | 3 | O | O | C₂H₅ | SC₂H₅ | CH₃ | 105 |
| 92 | 3 | O | O | C₂H₅ | N(CH₃)₂ | CH₃ | 148 |
| 93 | 3 | O | O | C₂H₅ | C₂H₅ | 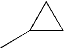 | 140 |
| 94 | 3 | O | O | C₂H₅ | C₃H₇-n | 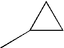 | 133 |
| 95 | 3 | O | O | C₂H₅ | C₃H₇-i | 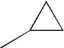 | 111 |
| 96 | 3 | O | O | C₂H₅ | SC₂H₅ | 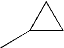 | 136 |
| 97 | 3 | O | O | C₂H₅ | N(CH₃)₂ | 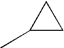 | 116 |
| 98 | 3 | O | O | C₂H₅ | C₂H₅ | CH₃ | 270 |
| | | | | | | | Na salt |
| 99 | 3 | O | O | C₂H₅ | OCH₃ | CH₃ | 203 |
| | | | | | | | Na salt |
| 100 | 3 | O | O | C₃H₇-n | OCH₃ | CH₃ | 127 |
| 101 | 3 | O | O | C₃H₇-n | OC₃H₇-i | CH₃ | 155 |
| 102 | 3 | O | O | C₃H₇-n | OCH₃ | 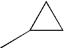 | 106 |
| 103 | 3 | O | O | C₃H₇-n | OC₃H₇-i | 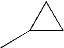 | 138 |
| 104 | 3 | O | O | C₃H₇-n | CH₃ | CH₃ | 302 |
| | | | | | | | Na salt |
| 105 | 3 | O | O | C₃H₇-n | C₃H₇-n | CH₃ | 261 |
| | | | | | | | Na salt |
| 106 | 3 | O | O | C₃H₇-n | C₃H₇-n | CH₃ | 93 |
| 107 | 3 | O | O | C₃H₇-n | CH₃ | CH₃ | 116 |
| 108 | 3 | O | O | C₃H₇-n | 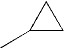 | CH₃ | 128 |
| 109 | 3 | O | O | C₃H₇-n | SC₂H₅ | CH₃ | 113 |
| 110 | 3 | O | O | C₃H₇-n | N(CH₃)₂ | CH₃ | 140 |
| 111 | 3 | O | O | C₃H₇-n | C₂H₅ | 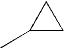 | 108 |
| 112 | 3 | O | O | C₃H₇-n | C₃H₇-n | 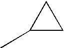 | 103 |
| 113 | 3 | O | O | C₃H₇-n | C₃H₇-i | 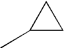 | 91 |
| 114 | 3 | O | O | C₃H₇-n | N(CH₃)₂ | 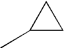 | 130 |
| 115 | 3 | O | O | C₃H₇-n | SC₂H₅ | 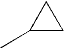 | 130 |
| 116 | 3 | O | O | C₃H₇-n | OC₂H₅ | CH₃ | 91 |
| 117 | 3 | O | O | C₃H₇-n | OC₃H₇-n | CH₃ | 106 |
| 118 | 2 | O | O | CH₃ | C₂H₅ | CH₃ | 109 |
| 119 | 2 | O | O | CH₃ | C₃H₇-n | CH₃ | 280 |
| | | | | | | | Na salt |
| 120 | 2 | O | O | CH₃ | 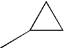 | CH₃ | 157 |
| 121 | 2 | O | O | CH₃ | CH₂OCH₃ | CH₃ | 106 |
| 122 | 2 | O | O | CH₃ | SCH₃ | CH₃ | 135 |
| 123 | 2 | O | O | CH₃ | N(CH₃)₂ | CH₃ | 141 |
| 124 | 2 | O | O | CH₃ | C₂H₅ | OC₂H₅ | 129 |
| 125 | 2 | O | O | CH₃ | CH₃ | 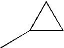 | 169 |
| 126 | 2 | O | O | CH₃ | OC₂H₅ | 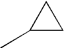 | 125 |
| 127 | 2 | O | O | CH₃ | OC₃H₇-n | 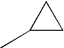 | 117 |
| 128 | 2 | O | O | CH₃ | 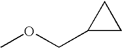 | CH₃ | 145 |
| 129 | 2 | O | O | CH₃ | 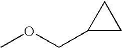 | 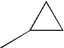 | 118 |
| 130 | 2 | O | O | CH₃ | C₃H₇-n | CH₃ | 108 |
| 131 | 2 | O | O | CH₃ | OCH₃ | CH₃ | 234 |
| | | | | | | | Na salt |
| 132 | 2 | O | O | CF₃ | OCH₃ | CH₃ | 185 |
| 133 | 2 | O | O | CF₃ | OC₂H₅ | CH₃ | |
| 134 | 2 | O | O | CF₃ | OC₂H₅ | 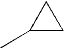 | 168 |
| 135 | 2 | O | O | CF₃ | 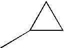 | 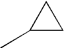 | 163 |
| 136 | 2 | O | O | CF₃ | OC₃H₇-n | CH₃ | 140 |
| 137 | 2 | O | O | CF₃ | OC₃H₇-i | CH₃ | 177 |
| 138 | 2 | O | O | CF₃ | OC₃H₇-n | 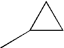 | 174 |
| 139 | 2 | O | O | CF₃ | OC₃H₇-i | 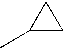 | 186 |
| 140 | 2 | O | O | CF₃ | CH₃ | CH₃ | 135 |
| 141 | 2 | O | O | CF₃ | SCH₃ | CH₃ | 186 |
| 142 | 2 | S | O | CF₃ | CH₃ | CH₃ | 103 |
| 143 | 2 | S | O | CF₃ | C₂H₅ | CH₃ | 137 |
| 144 | 2 | S | O | CF₃ | SCH₃ | CH₃ | 146 |
| 145 | 2 | S | O | CF₃ | OCH₃ | CH₃ | 156 |
| 146 | 2 | S | O | CF₃ | OC₂H₅ | CH₃ | 126 |
| 147 | 2 | S | O | CF₃ | OC₃H₇-n | CH₃ | 137 |
| 148 | 2 | S | O | CF₃ | OC₃H₇-i | CH₃ | 141 |
| 149 | 2 | S | O | CF₃ | OC₂H₅ | 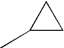 | 147 |
| 150 | 2 | S | O | CF₃ | OC₃H₇-i | 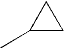 | |
| 151 | 2 | S | O | CF₃ | OCH₃ | 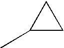 | 167 |
| 152 | 2 | S | O | CF₃ | OC₃H₇-n | 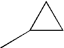 | 159 |
| 153 | 2 | S | O | CF₃ | 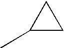 | 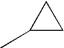 | 86 |
| 154 | 3 | S | O | C₂H₅ | OC₂H₅ | CH₃ | 134 |
| 155 | 3 | S | O | CH₃ | OC₂H₅ | CH₃ | 168 |
| 156 | 3 | S | O | C₃H₇-n | OC₂H₅ | CH₃ | 154 |
| 157 | 3 | S | O | C₂H₅ | OC₂H₅ | 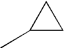 | 139 |
| 158 | 3 | S | O | CH₃ | OC₂H₅ | 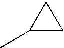 | 152 |
| 159 | 3 | S | O | C₂H₅ | OCH₃ | CH₃ | 171 |
| 160 | 3 | S | O | CH₃ | OCH₃ | CH₃ | 142 |
| 161 | 3 | S | O | C₂H₅ | OCH₃ | 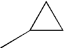 | |
| 162 | 3 | S | O | CH₃ | OCH₃ | 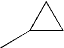 | |
| 163 | 3 | O | O | CF₃ | OCH₃ | CH₃ | 163 |
| 164 | 3 | O | O | CF₃ | OC₂H₅ | CH₃ | 131 |
| 165 | 3 | O | O | CF₃ | OC₃H₇-n | CH₃ | 120 |
| 166 | 3 | O | O | CF₃ | OCH₃ | 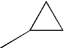 | 172 |
| 167 | 3 | O | O | CF₃ | OC₂H₅ | 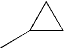 | 156 |
| 168 | 3 | O | O | CF₃ | OC₃H₇-n | 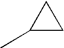 | 125 |
| 169 | 3 | O | O | CF₃ | OC₃H₇-i | 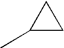 | 147 |
| 170 | 3 | O | O | CF₃ | 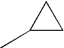 | 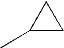 | 176 |
| 171 | 3 | O | O | CF₃ | CH₃ | CH₃ | 138 |
| 172 | 3 | O | O | CF₃ | SCH₃ | CH₃ | 139 |
| 173 | 3 | O | O | CF₃ | SC₂H₅ | CH₃ | 126 |
| 174 | 3 | O | O | CF₃ | OC₂H₅ | C₂H₅ | 130 |
| 175 | 3 | O | O | CF₃ | C₂H₅ | OC₂H₅ | 131 |
| 176 | 3 | O | O | CF₃ | OCH₂CF_{3 CH}₃ | 145 | |
| 177 | 3 | O | O | CF₃ | C₃H₇-n | OCH₃ | 155 |
| 178 | 3 | O | O | CF₃ | OCH₂CF₃ | 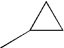 | 140 |
| 179 | 3 | S | O | CF₃ | OCH₃ | CH₃ | 164 |
| 180 | 3 | S | O | CF₃ | OC₂H₅ | CH₃ | 148 |
| 181 | 3 | S | O | CF₃ | OC₃H₇-n | CH₃ | 118 |
| 182 | 3 | S | O | CF₃ | OC₃H₇-i | CH₃ | 138 |
| 183 | 3 | S | O | CF₃ | OCH₃ | 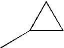 | 149 |
| 184 | 3 | S | O | CF₃ | OC₂H₅ | 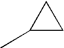 | 154 |
| 185 | 3 | S | O | CF₃ | OC₃H₇-n | 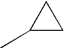 | 154 |
| 186 | 3 | S | O | CF₃ | OC₃H₇-i | 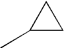 | 145 |

Starting Materials of the Formula (II):

### Example (II-1)

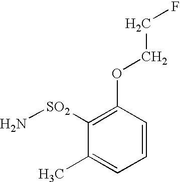

A mixture of 11.2 g (60 mmol) of 2-hydroxy-6-methyl-benzenesulphonamide, 10 g (78 mmol) of 1-bromo-2-fluoro-ethane, 16.6 g (120 mmol) of potassium carbonate and 350 ml of acetone is heated under reflux for 48 hours, and the hot mixture is then filtered. The filtrate is concentrated under reduced pressure and the residue is taken up in methylene chloride, washed with water, dried with sodium sulphate and filtered. The filtrate is concentrated, the residue is digested with diethyl ether and the resulting crystalline product is isolated by filtration with suction.

This gives 7.9 g (56% of theory) of 2-(2-fluoro-ethoxy)-6-methyl-benzenesulphonamide of melting point 103° C.

Analogously to Example (II-1), it is also possible to prepare, for example, the compounds of the general formula (II) listed in Table 2 below.

TABLE 2
Examples of the compounds of the formula (II)

| | |
|---|---|
| | (II) |
| 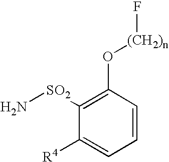 | |

| Ex. No. | n | R⁴ | Melting point (° C.) |
|---|---|---|---|
| II-2 | 2 | C₂H₅ | 108 |
| II-3 | 2 | CF₃ | 158-161 |
| II-4 | 2 | C₃H₇-i | 114 |
| II-5 | 2 | OC₂H₅ | 140 |
| II-6 | 2 | C₃H₇-n | 117 |
| II-7 | 3 | C₃H₇-n | 108 |
| II-8 | 2 | Br | |
| II-9 | 3 | C₂H₅ | 92 |
| II-10 | 3 | CH₃ | 118 |
| II-11 | 3 | C₃H₇-i | |
| II-12 | 3 | CF₃ | |
| II-13 | 3 | Br | |
| II-14 | 3 | OCH₃ | |
| II-15 | 2 | OCH₃ | |
| II-16 | 2 | OC₃H₇-n | |
| II-17 | 2 | OC₃H₇-i | |
| II-18 | 3 | OC₂H₅ | |
| II-19 | 3 | OC₃H₇-n | |
| II-20 | 3 | OC₃H₇-i | |
| II-21 | 2 | CH₂CF₃ | |
| II-22 | 3 | CH₂CF₃ | |
| II-23 | 2 | Cl | |
| II-24 | 3 | Cl | |
| II-25 | 2 | Br | |
| II-26 | 3 | Br | |
| II-27 | 2 | COOCH₃ | |
| II-28 | 3 | COOCH₃ | |
| II-29 | 2 | OCH₂CH₂F | |
| II-30 | 3 | OCH₂CH₂F | |
| II-31 | 2 | OCF₃ | |
| II-32 | 3 | OCF₃ | |
| II-33 | 3 | OCH₂CH₂CH₂F | |

### Example A

Pre-Emergence Test

Solvent: 5 parts by weight of acetone

Emulsifier: 1 part by weight of alkylaryl polyglycol ether

To produce a suitable preparation of active compound, 1 part by weight of active compound is mixed with the stated amount of solvent, the stated amount of emulsifier is added and the concentrate is diluted with water to the desired concentration.

Seeds of the test plants are sown in normal soil. After 24 hours, the soil is sprayed with the preparation of active compounds such that the particular amount of active compound desired is applied per unit area. The concentration of active compound in the spray liquor is chosen such that the particular amount of active compound desired is applied in 1000 litres of water per hectare.

After three weeks, the degree of damage to the plants is rated in % damaged in comparison to the development of the untreated control. The figures denote:

0%=no effect (like untreated control)

100%=total destruction

In this case, for example, the compounds of Preparation Examples 32, 34, 41, 93 and 114 show very strong action against weeds, and some of them are tolerated well by crop plants, such as, for example, sugar beet, barley, wheat and soya bean.

TABLE A-1

| pre-emergence | g ai/ha | Soya bean | Alopecurus | Amaranthus | Matricaria | Solanum | Viola |
|---|---|---|---|---|---|---|---|
| Ex. 32 | 8 | | 100 | 100 | 100 | 100 | 100 |

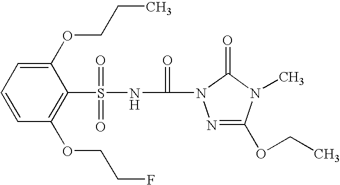

TABLE A-2

| pre-emergence | g ai/ha | Barley | Wheat | Alopecurus | Amaranthus | Matricaria | Solanum | Viola |
|---|---|---|---|---|---|---|---|---|
| Ex. 34 | 4 | 10 | 0 | 100 | 95 | 100 | 95 | 95 |

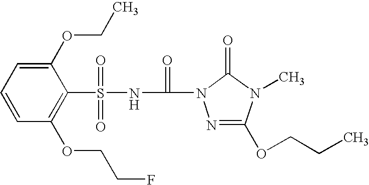

TABLE A-3

| pre-emergence | g ai/ha | Wheat | Echinochloa | Lolium | Amaranthus | Galium | Matricaria | Stellaria |
|---|---|---|---|---|---|---|---|---|
| Ex. 41 | 30 | 0 | 95 | 90 | 95 | 100 | 100 | 100 |

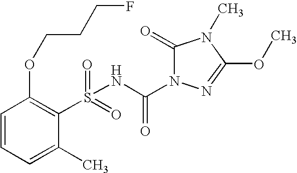

TABLE A-4

| pre-emergence | g ai/ha | Alopecurus | Echinochloa | Lolium | Amaranthus | Galium | Stellaria | Viola |
|---|---|---|---|---|---|---|---|---|
| Ex. 93 | 30 | 95 | 100 | 95 | 100 | 95 | 100 | 100 |

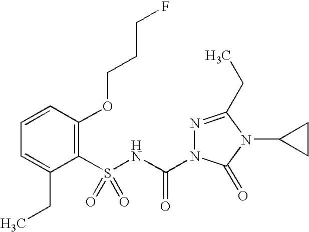

TABLE A-5

| pre-emergence | g ai/ha | Sugar beet | Alopecurus | Lolium | Amaranthus | Galium | Stellaria |
|---|---|---|---|---|---|---|---|
| Ex. 114 | 30 | 0 | 95 | 95 | 95 | 80 | 95 |

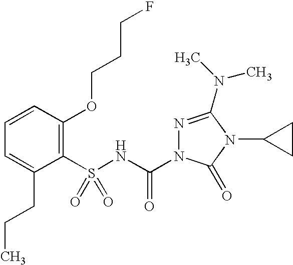

### Example B

Post-Emergence Test

Solvent: 5 parts by weight of acetone

Emulsifier: 1 part by weight of alkylaryl polyglycol ether

To produce a suitable preparation of active compound, 1 part by weight of active compound is mixed with the stated amount of solvent, the stated amount of emulsifier is added and the concentrate is diluted with water to the desired concentration.

Test plants of a height of 5-15 cm are sprayed with the preparation of active compound such that the particular amounts of active compound desired are applied per unit area. The concentration of the spray liquor is chosen such that the particular amounts of active compound desired are applied in 10001 of water/ha.

After three weeks, the degree of damage to the plants is rated in % damage in comparison to the development of the untreated control.

The figures denote:

0%=no effect (like untreated control)

100%=total destruction

In this test, for example, the compounds of Preparation Examples 32, 33, 90, 97 and 111 show very strong action against weeds, and some of them are tolerated well by crop plants, such as, for example, maize, wheat and sugar beet.

TABLE B-1

| post-emergence | g ai/ha | Setaria | Abutilon | Amaranthus | Matricaria | Stellaria | Xanthium |
|---|---|---|---|---|---|---|---|
| Ex. 32 | 15 | 95 | 100 | 100 | 100 | 100 | 100 |

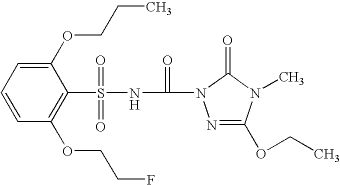

TABLE B-2

| post-emergence | g ai/ha | Alopecurus | Echinochloa | Lolium | Abutilon | Amaranthus | Matricaria | Stellaria |
|---|---|---|---|---|---|---|---|---|
| Ex. 33 | 15 | 95 | 100 | 95 | 100 | 100 | 100 | 100 |

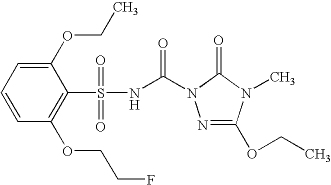

TABLE B-4

| post-emergence | g ai/ha | Maize | Alopecurus | Amaranthus | Ipomoea | Solanum | Stellaria |
|---|---|---|---|---|---|---|---|
| Ex. 90 | 30 | 10 | 90 | 100 | 90 | 95 | 100 |

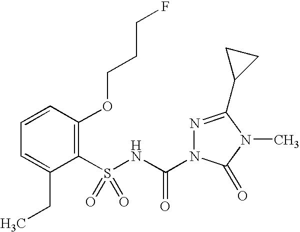

TABLE B-5

| | | Sugar | | | | | | |
|---|---|---|---|---|---|---|---|---|
| post-emergence | g ai/ha | beet | Alopecurus | Avena fatua | Echinochloa | Amaranthus | Solanum | Stellaria |
| Ex. 97 | 30 | 10 | 90 | 90 | 95 | 100 | 90 | 100 |
| 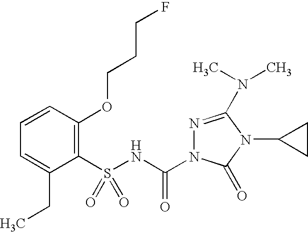 | | | | | | | | |

TABLE B-6

| post-emergence | g ai/ha | Wheat | Avena fatua | Echinochloa | Amaranthus | Ipomoea | Stellaria | Viola |
|---|---|---|---|---|---|---|---|---|
| Ex. 111 | 30 | 0 | 90 | 90 | 100 | 100 | 100 | 95 |
| 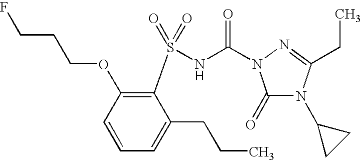 | | | | | | | | |

1. Compounds of the general formula (I) 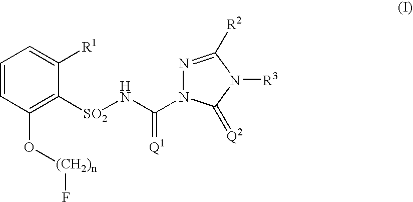
in which
n represents the numbers 2, 3 or 4,
Q¹ represents O (oxygen) or S (sulphur),
Q² represents O (oxygen) or S (sulphur),
R¹ represents in each case optionally cyano-, fluorine-, chlorine-, bromine-, methoxy-, ethoxy-, n- or i-propoxy-substituted methyl, ethyl, n- or i-propyl, n-, i-, s- or t-butyl, methoxy, ethoxy, n- or i-propoxy, n-, i-, s- or t-butoxy, methoxycarbonyl, ethoxycarbonyl, n- or i-propoxycarbonyl,
R² represents hydrogen, cyano, fluorine, chlorine, bromine, represents in each case optionally cyano-, fluorine-, chlorine-, methoxy-, ethoxy-, n- or i-propoxy-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethyl amino, n- or i-propylamino, n-, i-, s- or t-butylamino, methoxyamino, ethoxyamino, n- or i-propoxyamino, n-, i-, s- or t-butoxyamino, represents dimethylamino, diethylamino, N-methyl-methoxyamino or N-methyl-ethoxyamino, represents in each case optionally cyano-, fluorine-, chlorine- or bromine-substituted ethenyl, propenyl, butenyl, ethinyl, propinyl, butinyl, propenyloxy, butenyloxy, propinyloxy, butinyloxy, propenylthio, butenylthio, propinylthio, butinylthio, propenylamino, butenylamino, propinylamino or butinylamino, or represents in each case optionally cyano-, fluorine-, chlorine-, bromine-, methyl- or ethyl-substituted cyclopropyl, cyclobutyl, cyclopentyl, cyclohexyl, cyclopropyloxy, cyclobutyloxy, cyclopentyloxy, cyclohexyloxy, cyclopropylamino, cyclobutylamino, cyclopentylamino, cyclohexylamino, cyclopropylmethyl, cyclobutylmethyl, cyclopentylmethyl, cyclohexylmethyl, cyclopropylmethoxy, cyclobutylmethoxy, cyclopentylmethoxy, cyclohexylmethoxy, cyclopropylmethylthio, cyclobutylmethylthio, cyclopentylmethylthio, cyclohexylmethylthio, cyclopropylmethylamino, cyclobutylmethylamino, cyclopentylmethylamino or cyclohexylmethylamino, and
R³ represents hydrogen, represents in each case optionally cyano-, fluorine-, chlorine-, methoxy-, ethoxy-, n- or i-propoxy-substituted methyl, ethyl, n- or i-propyl, methoxy, ethoxy, n- or i-propoxy, methylamino, ethylamino, n- or i-propylamino, represents dimethylamino or diethylamino, or represents in each case optionally cyano-, fluorine-, chlorine-, bromine-, methyl- or ethyl-substituted cyclopropyl, cyclobutyl, cyclopentyl, cyclohexyl, cyclopropylmethyl, cyclobutylmethyl, cyclopentylmethyl or cyclohexylmethyl,
and also salts of compounds of the formula (I),
except for the compounds 2-(2-fluoro-ethoxy)-6-methyl-N-[(4-methyl-5-oxo-3-propoxy-4,5-dihydro-1H-1,2,4-triazol-1-yl)-carbonyl]-benzenesulphonamide, 2-(2-fluoro-ethoxy)-6-methyl-N-[(4-methyl-5-oxo-3-1-propoxy-4,5-dihydro-1H-1,2,4-triazol-1-yl)-carbonyl]-benzenesulphonamide, 2-(2-fluoroethoxy)-6-methyl-N-[4-cyclopropyl-3-methoxy-5-oxo-4,5-dihydro-1H-1,2,4-triazol-1-yl)-carbonyl]-benzenesulphonamide, 2-(2-fluoro-ethoxy)-6-methyl-N-[4-cyclopropyl-5-oxo-3-1-propoxy-4,5-dihydro-1H-1,2,4-triazol-1-yl)-carbonyl]-benzenesulphonamide, 2-(2-fluoro-ethoxy)-6-methyl-N-[3-methoxy-4-methyl-5-oxo-4,5-dihydro-1H-1,2,4-triazol-1-yl)-carbonyl]-benzenesulphonamide, 2-(2-fluoro-ethoxy)-6-methyl-N-[3-ethoxy-4-methyl-5-oxo-4,5-dihydro-1H-1,2,4-triazol-1-yl)-carbonyl]-benzenesulphonamide and 2-(2-fluoro-ethoxy)-6-methyl-N-[3,4-dimethyl-5-oxo-4,5-dihydro-1H-1,2,4-triazol-1-yl)-carbonyl]-benzenesulphonamide, which are excluded by disclaimer.

2. Compounds according to claim 1, characterized in that
n represents the numbers 2, 3 or 4,
Q¹ represents O (oxygen) or S (sulphur),
Q² represents O (oxygen) or S (sulphur),
R¹ represents in each case optionally cyano-, fluorine-, chlorine-, methoxy- or ethoxy-substituted methyl, ethyl, n- or i-propyl, n- or i-butyl, methoxy, ethoxy, n- or i-propoxy, n- or i-butoxy, methoxycarbonyl, ethoxycarbonyl, n- or i-propoxycarbonyl,
R² represents hydrogen, cyano, fluorine, chlorine, bromine, represents in each case optionally cyano-, fluorine-, chlorine-, methoxy-, ethoxy-, n- or i-propoxy-substituted methyl, ethyl, n- or i-propyl, n-, i- or s-butyl, methoxy, ethoxy, n- or i-propoxy, n-, i- or s-butoxy, methylthio, ethylthio, n- or i-propylthio, n-, i- or s-butylthio, methylamino, ethylamino, n- or i-propylamino, n-, i- or s-butylamino, methoxyamino, ethoxyamino, n- or i-propoxyamino, n-, i- or s-butoxyamino, represents dimethylamino, diethylamino, N-methyl-methoxyamino or N-methyl-ethoxyamino, represents in each case optionally cyano-, fluorine-, chlorine- or bromine-substituted ethenyl, propenyl, butinyl, ethinyl, propinyl, butinyl, propenyloxy, butenyloxy, propinyloxy, butinyloxy, propenylthio, butenylthio, propinylthio, butinylthio, propenylamino, butenylamino, propinylamino or butinylamino, or represents in each case optionally cyano-, fluorine-, chlorine-, bromine-, methyl- or ethyl-substituted cyclopropyl, cyclobutyl, cyclopentyl, cyclopropyloxy, cyclobutyloxy, cyclopentyloxy, cyclopropylamino, cyclobutylamino, cyclopentyl amino, cyclopropylmethyl, cyclobutylmethyl, cyclopentylmethyl, cyclopropylmethoxy, cyclobutylmethoxy, cyclopentylmethoxy, cyclopropylmethylthio, cyclobutylmethylthio, cyclopentylmethylthio, cyclopropylmethylamino, cyclobutylmethyl amino or cyclopentylmethylamino, and
R³ represents hydrogen, represents in each case optionally cyano-, fluorine-, chlorine-, methoxy- or ethoxy-substituted methyl, ethyl, methoxy, ethoxy, methylamino, ethylamino, represents dimethylamino, or represents in each case optionally fluorine-, chlorine- or methyl-substituted cyclopropyl, cyclobutyl, cyclopentyl, cyclopropylmethyl, cyclobutylmethyl or cyclopentylmethyl.

3. Compounds according to claim 1, characterized in that
n represents the numbers 2, 3 or 4,
Q¹ represents O (oxygen) or S (sulphur),
Q² represents O (oxygen) or S (sulphur),
R¹ represents in each case optionally fluorine-, chlorine-, methoxy- or ethoxy-substituted methyl, ethyl, n- or i-propyl, methoxy, ethoxy, n- or i-propoxy, methoxycarbonyl or ethoxycarbonyl,
R² represents chlorine, bromine, represents in each case optionally cyano-, fluorine-, chlorine-, methoxy-, ethoxy-, n- or i-propoxy-substituted methyl, ethyl, n- or i-propyl, methoxy, ethoxy, n- or i-propoxy, methylthio, ethylthio, n- or i-propylthio, methylamino, ethylamino, n- or i-propylamino, methoxyamino or ethoxyamino, represents dimethylamino or N-methyl-methoxyamino, represents in each case optionally fluorine- or chlorine-substituted ethenyl, propenyl, ethinyl, propinyl, propenyloxy, propinyloxy, propenylthio, propinylthio, propenylamino or propinylamino, or represents in each case optionally fluorine-, chlorine- or methyl-substituted cyclopropyl, cyclopropyloxy, cyclopropylamino, cyclopropylmethyl, cyclopropylmethoxy, cyclopropylmethylthio or cyclopropylmethylamino, and
R³ represents methyl, ethyl, methoxy, ethoxy, dimethylamino, cyclopropyl or cyclopropylmethyl.

4. Compounds according to claim 1 in which R¹ does not represent methyl if n represents 2.

5. Compounds according to claim 1 in which n represents 3 or 4.

6. Process for preparing compounds according to claim 1, characterized in that
(a) fluoroalkoxybenzenesulphonamides of the general formula (II) 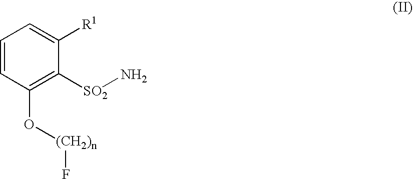
in which
n and R¹ are as defined in claim 1
are reacted with substituted (thio)carbonyl-triazolin(ethi)ones of the general formula (III) 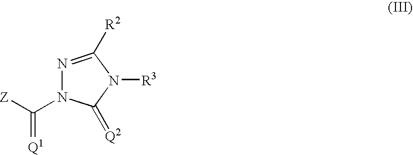
in which
Q¹, Q², R² and R³ are as defined in claim 1 and
Z represents halogen or represents in each case optionally substituted alkoxy, aryloxy or arylalkoxy,
if appropriate in the presence of one or more reaction auxiliaries and if appropriate in the presence of one or more diluents,
or that
(b) fluoroalkoxyphenylsulphonyl iso(thio)cyanates of the general formula (IV) 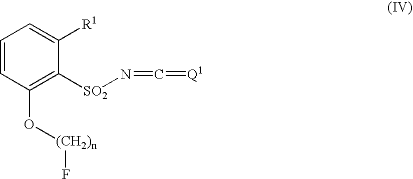
in which
n, Q¹ and R¹ are as defined in claim 1
are reacted with triazolin(ethi)ones of the general formula (V) 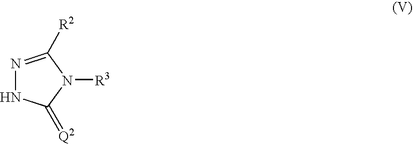
in which
Q², R² and R³ are as defined in claim 1,
if appropriate in the presence of one or more reaction auxiliaries and if appropriate in the presence of one or more diluents,
or that
(c) fluoroalkoxybenzenesulphonyl chlorides of the general formula (VI) 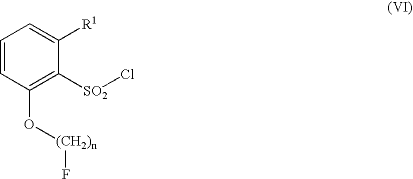
in which
n and R¹ are as defined in claim 1
are reacted with triazolin(ethi)ones of the general formula (V) 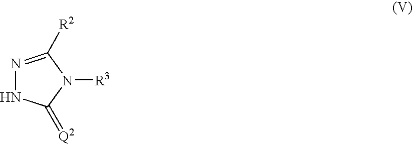
in which
Q², R² and R³ are as defined in claim 1
and metal (thio)cyanates of the general formula (VII)
MQ¹CN (VII)
in which
Q¹ is as defined in claim 1 and
M represents a metal equivalent,
if appropriate in the presence of one or more reaction auxiliaries and if appropriate in the presence of one or more diluents,
and the compounds of the general formula (I) obtained by process (a), (b) or (c) are, if appropriate, converted into salts using customary methods.

7. Method for controlling undesirable vegetation, characterized in that at least one compound according to any of claims 1 to 5 is allowed to act on the undesirable plants and/or their habitat.

8. Use of at least one compound according to any of claims 1 to 5 for controlling undesirable plants.

9. Herbicidal composition, characterized in that it comprises a compound according to any of claims 1 to 5 and customary extenders and/or surfactants.
